(19) United States
(12) Patent Application Publication
(10) Pub. No.: US 2007/0004002 A1
(43) Pub. Date: Jan. 4, 2007

(54) Artificial mammalian chromosome

(75) Inventor: Tsuneko Okazaki, AICHI (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: JAPAN SCIENCE AND TECHNOLOGY AGENCY, SAITAMA (JP)

(21) Appl. No.: 10/526,425

(22) PCT Filed: Sep. 1, 2003
(86) PCT No.: PCT/JP03/11134

§ 371 Date: Mar. 3, 2005

(30) Foreign Application Priority Data
Sep. 3, 2002 (JP) 2002-258114
Nov. 22, 2002 (JP) 2002-338865

(51) Int. Cl. C12P 21/06; C12N 15/09; C07K 14/80; C07H 21/04

(52) U.S. Cl. 435/69.1; 435/455; 435/320.1; 435/325; 530/383; 536/23.5

(57) ABSTRACT

It is intended to provide an artificial mammalian chromosome which is stably held in mammalian cells and allows efficient expression of a target gene carried thereby. Namely, a first cyclic vector containing a mammalian centromere sequence and a selection marker gene and a second cyclic vector containing a functional sequence are transferred into mammalian host cells. Then transformed cells are selected by using the above-described selection marker gene and cells holding an artificial mammalian chromosome are selected from among the transformed cells thus selected. Thus, it is possible to construct an artificial mammalian chromosome which has a mammalian replication origin, the mammalian centromere sequence and the functional sequence, is in a cyclic form, can be replicated in mammalian cells, extrachromosomally held in the host cells and transferred to daughter cells in cell division.

### TECHNICAL FIELD

The present invention relates to a mammalian artificial chromosome. More particularly, the present invention relates to a production method of a mammalian artificial chromosome, a mammalian artificial chromosome and a use of a mammalian artificial chromosome. The mammalian artificial chromosome provided in the present invention can be used, for example, as a vector to carry a gene of interest to mammalian cells for gene therapy, transformation of cells, tissues or individual bodies of mammalian, and the like.

### BACKGROUND ART

Mitotically stable human artificial chromosomes (HACs), several mega-base pairs in size, are frequently generated de novo in the human fibroblast cell line, HT1080, upon introduction of precursor DNA constructs in either linear (YAC) or circular form (BAC or PAC) containing several tens of kilo-bases of human alpha-satellite (alphoid) DNA with frequent CENP-B boxes (Ikeno et al. 1998; Henning et al. 1999; Ebersole et al. 2000). Since essential kinetochore proteins are detected on such HACs, the input alpha-satellite arrays are capable of assembling a de novo active centromere/kinetochore structure similar to that of authentic human chromosomes (Ikeno et al. 1994; Ikeno et al. 1998; Henning et al. 1999; Ebersole et al. 2000; Ando et al. 2002). Since HACs duplicate once every cell cycle utilizing cellular protein factors, they also contain replication origin(s) in the alphoid sequence. Linear HACs made from the alphoid-YAC with telomere sequences acquired a functional telomere structure at the ends of the HACs, but circular HACs made from BAC or PAC had no telomere structure (Ikeno et al. 1998; Ebersole et al. 2000).

Treating human diseases by gene therapy is a challenging and promising field. Although we now have at hand tens of thousands of genes by which we might be able to cure defective human genes or to characterize in detail their function and regulation, the major obstacle still lies in the development of effective gene delivery technology. Presently available vectors for mammalian cells are mainly derived from small viruses (Mineta et al. 1995; Fisher et al. 1997; Pfeiter & Verna 2001). Although they have the advantage of highly efficient transduction of the genes of interest (transgenes), their cloning capacity is limited. They are too small to include large genome segments with tissue-specific regulatory regions. Moreover, transgenes are usually maintained stably only after random integration into host-cell chromosomes, the gene expression from which is usually unpredictable (mostly suppressed) and not under the control of the authentic regulatory region of the genes. Even worse, the step might induce unfavorable mutagenesis.

In contrast, HACs have the capacity to accommodate a large transgene with a controlling region in excess of 100 kb of DNA. HACs containing transgenes are generated de novo from a precursor construct with both the transgene and an alphoid array (Mejia et al. 2001) or from precursor constructs containing an alphoid array and the transgene in separate entities (Grimes et al. 2001). Thus, HACs may be used not only as vectors in therapeutic applications but also as model systems useful in the analysis of tissue or organ specific regulation of gene expression that is only possible with large genome segments.

### DISCLOSURE OF THE INVENTION

The present invention has been made under the above-mentioned circumstances. It is an object of the present invention to provide a technology for stably expressing a targeted functional sequence of a gene, etc. in a mammalian cell. Specifically, it is an object of the present invention to provide a mammalian artificial chromosome which is stably maintained in a mammalian cell and is capable of efficiently expressing a functional sequence contained therein, a production method of the same, and a method of transforming cells etc. by using the same, and the like.

The present inventors have considered the objects mentioned above and have attempted to produce a mammalian artificial chromosome containing a target gene (GCH1 gene) in a state of being capable of expressing by employing a method of taking the target gene as a functional sequence during a process in which the mammalian artificial chromosome is formed from a precursor of an artificial chromosome. That is to say, the present inventors used BAC that is a circular vector as a artificial chromosome precursor, and co-transfected BAC (GCH1-BAC) containing about 180 kb of a genome region covering an entire GCH1 gene and its upstream regulatory region and BAC (alphoid BAC) including about 50 kb or about 100 kb of an alphoid array as a human centromere sequence with HT1080 cell, which is a human fibroblast cell. As a result, we successfully obtained a human artificial chromosome (HAC) having plural copies of GCH1 genes. It was shown that the HAC obtained was able to be maintained stably in both human cells and mouse cells even if selection operation is not carried out. When a further investigation was carried out, the increase in the GCH1 activity was observed in the transformed cell lines having the HAC and the activity showed the response with respect to the induction of interferon γ as in the case that is present on the chromosome. That is to say, the natural expression of a GCH1 gene from the constructed HAC was confirmed.

Meanwhile, the present inventors have succeeded, by using a linear vector YAC as a precursor, in constructing a human artificial chromosome containing an entire region of human β globin gene cluster by the same method as in the case of BAC.

Furthermore, the present inventors have succeeded in transferring the constructed HAC into mouse embryonic stem cells (ES cells) and creating a chimeric mouse (HAC-containing mouse) by using the obtained ES cells. This is extremely significant that it was experimentally confirmed that an artificial chromosome could be used as a tool for gene introduction at the individual body level. Furthermore, the present inventors succeeded in transferring HAC into not only XY nuclear type ES cells but also XO nuclear type ES cells and further in creating a female chimeric mouse containing HAC by the use of the same. Note here that it is thought that the use of female chimeric mice makes it easy to transmit a mammalian artificial chromosome.

Furthermore, in the production of a mammalian artificial chromosome having a gene insertion site, when a mammalian artificial chromosome was constructed by inserting an insulator sequence for the purpose of promoting the expression of gene to be introduced later, surprisingly, the efficiency of gene transfer into the mammalian artificial chromosome was enhanced. In other words, it was found that the use of the insulator sequence makes it possible to produce efficiently mammalian artificial chromosome having a target gene.

The present invention was made based on the findings in the above-mentioned investigation and the present invention provides the following configurations.

[1] A production method of a mammalian artificial chromosome, comprising:

a first step of introducing a first vector being circular in form and comprising a mammalian centromere sequence and a second vector being circular in form and comprising a functional sequence into a mammalian host cell;

a second step of selecting transformed cells; and

a third step of selecting a cell containing a mammalian artificial chromosome from the selected transformed cells.

[2] A production method of a mammalian artificial chromosome, comprising:

a first step of introducing a first vector consisting of a yeast artificial chromosome having a mammalian centromere sequence and a mammalian telomere sequence and a second vector consisting of a yeast artificial chromosome having a functional sequence into a mammalian host cell;

a second step of selecting transformed cells; and

a third step of selecting a cell containing a mammalian artificial chromosome from the selected transformed cells.

[3] The production method according to 1 or 2, wherein the first vector has a selection marker gene and the selection of the transformed cells in the second step is carried out by using the selection marker gene.

[4] The production method according to any of 1 to 3, wherein the mammalian centromere sequence comprises a region in which a plurality of the following sequences are arranged at regular intervals:

5′-NTTCGNNNNANNCGGGN-3′: SEQ ID NO. 1, wherein N is selected from the group consisting of A, T, C and G.

[5] The production method according to any one of 1 to 4, wherein the mammalian centromere sequence comprises a sequence derived from a human chromosome alpha satellite region.

[6] The production method according to 5, wherein the mammalian centromere sequence comprises a 11mer repeat unit derived from a human chromosome 21.

[7] The production method according to any of 1 to 6, wherein the size of the mammalian centromere sequence is about 50 kb or less.

[8] The production method according to any of 1 to 7, wherein the functional sequence consists of a sequence encoding a target gene and a regulatory region thereof.

[9] The production method according to 8, wherein the target gene is a gene other than housekeeping genes.

[10] The production method according to 8, wherein the target gene is a structural gene of human guanosine triphosphate cyclohydrolase I.

[11] The production method according to 8, wherein the functional sequence is a sequence encoding an entire region of a human β globin gene cluster.

[12] The production method according to any of 1 to 7, wherein the functional sequence consists of an insertion sequence for specifically inserting a sequence of interest.

[13] The production method according to 12, wherein the insertion sequence is a loxP site, a FRT site, or a sequence obtained by partial modification of a loxP site or a FRT site and has a function for inserting the sequence of interest.

[14] The production method according to any of 1 to 13, wherein the quantity ratio of the first vector to the second vector, which are inserted in the first step, is in the range from about 10:1 molecular ratio to about 1:10 molecular ratio.

[15] The production method according to any of 1 to 14, wherein a plurality of vectors comprising different functional sequences are used as the second vector.

[16] The production method according to any of 1 to 15, wherein the second vector further comprises an insulator sequence.

[17] A mammalian artificial chromosome obtainable by the production method described in any of 1 to 16,

which comprises a mammalian replication origin, a mammalian centromere sequence and a functional sequence; and

which is circular in form and is replicated in a mammalian cell, maintained extrachromosomally in a host cell, and transmitted to daughter cells during cell division.

[18] A mammalian artificial chromosome obtainable by the production method described in any of 1 to 16,

which comprises a mammalian replication origin, a mammalian centromere sequence, a mammalian telomere sequence, and a functional sequence encoding a target gene and a regulatory region thereof; and

which is linear in form and is replicated in a mammalian cell, maintained extrachromosomally in a host cell, and transmitted to daughter cells during cell division.

[19] A mammalian artificial chromosome,

which comprises a mammalian replication origin, a mammalian centromere sequence, and a functional sequence encoding a target gene (excluding a housekeeping gene) and a regulatory region thereof, and

which is circular in form and is replicated in a mammalian cell, maintained extrachromosomally in a host cell, and transmitted to daughter cells during cell division.

[20] The mammalian artificial chromosome according to 19, wherein the target gene is a structural gene of a human guanosine triphosphate cyclohydrolase I.

[21] A mammalian artificial chromosome,

which comprises a mammalian replication origin, a mammalian centromere sequence, a mammalian telomere sequence, and a functional sequence encoding a target gene (excluding a housekeeping gene) and a regulatory region thereof, and

which is linear in form and is replicated in a mammalian cell, maintained extrachromosomally in a host cell, and transmitted to daughter cells during cell division.

[22] The mammalian artificial chromosome according to 21, wherein the functional sequence consists of an entire region of a human β globin gene cluster.

[23] A mammalian artificial chromosome,

which comprises a mammalian replication origin, a mammalian centromere sequence, and an insertion sequence for specifically inserting a sequence of interest, and

which is circular in form and is replicated in a mammalian cell, maintained extrachromosomally in a host cell, and transmitted to daughter cells during cell division.

[24] A mammalian artificial chromosome,

which comprises a mammalian replication origin, a mammalian centromere sequence, a mammalian telomere sequence, and an insertion sequence for specifically inserting a sequence of interest,

which is linear in form and is replicated in a mammalian cell, maintained extrachromosomally in a host cell, and transmitted to daughter cells during cell division.

[25] The mammalian artificial chromosome according to 23 or 24, wherein the insertion sequence is a loxP site, a FRT site, or a sequence obtained by partial modification of a loxP site or a FRT site and has a function for inserting the sequence of interest.

[26] The mammalian artificial chromosome according to any of 17 to 25, wherein the mammalian centromere sequence comprises a region in which a plurality of the following sequences are arranged at regular intervals:

5′-NTTCGNNNNANNCGGGN-3′: SEQ ID NO. 1, wherein N is selected from the group consisting of A, T, C and G.

[27] The mammalian artificial chromosome according to any of 17 to 25, wherein the mammalian centromere sequence comprises a sequence derived from a human chromosome alpha satellite region.

[28] The mammalian artificial chromosome according to 27, wherein the mammalian centromere sequence comprises an 11mer repeat unit derived from a human chromosome 21.

[29] The mammalian artificial chromosome according to any of 17 to 28, comprising a plurality of the functional sequences or the insertion sequences.

[30] The mammalian artificial chromosome according to any of 17 to 29, further comprising an insulator sequence.

[31] A mammalian cell containing the mammalian artificial chromosome described in any of 17 to 30 outside the autonomous chromosome.

[32] A human cell containing the mammalian artificial chromosome described in any of 17 to 30 outside the autonomous chromosome.

[33] An embryonic stem cell containing the mammalian artificial chromosome described in any of 17 to 30 outside the autonomous chromosome.

[34] A production method of a mammalian cell in which the functional sequence or the insertion sequence is introduced in a state in which they can be maintained stably for a long term, the method comprising:

introducing the mammalian artificial chromosome obtained by the production method described in any of 1 to 16 or the mammalian artificial chromosome described in any of 17 to 30 into mammalian cells as target cells.

[35] A production method of a mammalian cell containing a mammalian artificial chromosome, the method comprising:

a first step of introducing a first vector being circular in form and comprising a mammalian centromere sequence and a second vector being circular in form and comprising a functional sequence into mammalian host cells;

a second step of selecting transformed cells;

a third step of selecting a cell containing a mammalian artificial chromosome from the selected transformed cells;

a fourth step of isolating the mammalian artificial chromosome from the selected cells; and

a fifth step of introducing the isolated mammalian artificial chromosome into a mammalian cell as a target cell.

[36] A production method of a mammalian cell containing a mammalian artificial chromosome, the method comprising:

a first step of introducing a first vector consisting of a yeast artificial chromosome having a mammalian centromere sequence and a mammalian telomere sequence and a second vector consisting of a yeast artificial chromosome having a functional sequence into mammalian host cells;

a second step of selecting transformed cells;

a third step of selecting a cell containing a mammalian artificial chromosome from the selected transformed cells;

a fourth step of isolating the mammalian artificial chromosome from the selected cell; and

a fifth step of introducing the isolated mammalian artificial chromosome into a mammalian cell as a target cell.

[37] A production method of a micro-cell containing a mammalian artificial chromosome, the method comprising:

a first step of introducing a first vector being circular in form and comprising a mammalian centromere sequence and a second vector being circular in form and comprising a functional sequence into mammalian host cells;

a second step of selecting transformed cells;

a third step of selecting a cell containing a mammalian artificial chromosome from the selected transformed cells;

a fourth step of fusing the selected cell with a mammalian cell having an ability of forming micro-cells;

a fifth step of selecting a hybrid cell capable of forming micro-cells and containing the mammalian artificial chromosome; and

a sixth step of forming micro-cells from the selected hybrid cell.

[38] A production method of a micro-cell containing a mammalian artificial chromosome, the method comprising:

a first step of introducing a first vector consisting of a yeast artificial chromosome including a mammalian centromere sequence and a mammalian telomere sequence and a second vector consisting of a yeast artificial chromosome including a functional sequence into mammalian host cells;

a second step of selecting transformed cells;

a third step of selecting a cell containing a mammalian artificial chromosome from the selected transformed cells;

a fourth step of fusing the selected cell with a mammalian cell having an ability of forming micro-cells;

a fifth step of selecting a hybrid cell having an ability of forming micro-cells and containing a mammalian artificial chromosome; and

a sixth step of forming micro-cells from the selected hybrid cell.

[39] A production method of mammalian cells containing a mammalian artificial chromosome, comprising:

fusing the micro-cell obtainable by the production method described in 37 or 38 with a mammalian cell as a target cell.

[40] A production method of a mammalian cell containing a mammalian artificial chromosome, comprising:

isolating the mammalian artificial chromosome from the host cell containing the mammalian artificial chromosome described in any of 17 to 30; and

introducing the isolated mammalian artificial chromosome into a mammalian cell as a target cell.

[41] A production method of a micro-cell containing a mammalian artificial chromosome, the method comprising:

fusing a host cell containing the mammalian artificial chromosome described in any of 17 to 30 and a mammalian cell having an ability of forming micro-cells;

selecting a hybrid cell having an ability of forming micro-cellsi and containing the mammalian artificial chromosome; and

forming micro-cells from the selected hybrid cells.

[42] A production method of a mammalian cell containing a mammalian artificial chromosome, the method comprising:

fusing the micro-cell obtainable by the production method described in 41 with a mammalian cell as a target.

[43] The production method of a mammalian cell according to any of 34, 35, 36, 39, 40 and 42, wherein the mammalian cell as a target cell is an embryonic stem cell, embryonic germ cell, or tissue stem cell.

[44] The production method of a mammalian cell according to any of 34, 35, 36, 39, 40 and 42, wherein the mammalian cell as a target cell is formed by inducing an embryonic stem cell, embryonic germ cell, or tissue stem cell so as to be differentiated to a cell of specific tissue.

[45] The production method of a mammalian cell according to any of 34, 35, 36, 39, 40 and 42, wherein the mammalian cell as a target cell is a fertilized egg.

[46] A vector used for producing a mammalian artificial chromosome, comprising a mammalian centromere sequence having the size of about 50 kb or less and a selection marker gene.

[47] The vector according to 46, wherein the mammalian centromere sequence comprises a region in which a plurality of the following sequences are arranged at regular intervals:

5′-NTTCGNNNNANNCGGGN-3′: SEQ ID NO. 1, wherein N is selected from the group consisting of A, T, C and G.

[48] The vector according to 46 or 47, wherein the mammalian centromere sequence comprises a sequence derived from a human chromosome alpha satellite region.

[49] The vector according to 48, wherein the mammalian centromere sequence comprises an 11mer repeat unit derived from a human chromosome 21.

[50] A vector used for producing a mammalian artificial chromosome, comprising: a sequence of a loxP site or FRT site, or a sequence obtainable by partial modification of a loxP site or FRT site, the sequence having a function for inserting the sequence of interest, and an insulator sequence.

[51] A non-human transformed animal into which a mammalian artificial chromosome is introduced.

[52] The non-human transformed animal according to 51, wherein the mammalian artificial chromosome is a mammalian artificial chromosome described in any of 17 to 19.

[53] An XO type mouse embryonic stem cell into which a mammalian artificial chromosome is introduced.

[54] The XO type mouse embryonic stem cell according to 53, wherein the mammalian artificial chromosome is a mammalian artificial chromosome described in any of 17 to 19.

[55] A female chimeric mouse into which a mammalian artificial chromosome is introduced.

[56] The female chimeric mouse according to 55, wherein the mammalian artificial chromosome is a mammalian artificial chromosome described in any of 17 to 19.

### BRIEF DESCRIPTION OF THE DRAWINGS

FIG. 1 is a table summarizing the fates of co-transfected BACs in the transformed cell lines. BS-resistant cell lines obtained by co-transfection of GCH1-BAC plus CMV/a100 BAC or SV/a50 BAC were analyzed by FISH. “HAC” indicates cell lines with an artificial chromosome detected both with a21-I alphoid DNA and BAC vector probes. One copy of HAC was detected in more than 95% of the inspected metaphase spread of these cell lines. In the remaining cell lines, introduced BACs were either integrated into chromosomes of HT1080 (Chromosome) or signals were undetectable (Non) by FISH analysis. “HAC with GCH” indicates the cell lines carrying a HAC with signals for the GCH1 gene.

FIG. 2 is a table summarizing GCH1 activity in HAC-containing cell lines. GCH1 activity of HT/GCH2-10, HT/GCH5-18 and HT1080 cells was measured in the presence or absence of IFN-γ induction. Data are mean+/−SD values from three independent experiments.

FIG. 3 shows constructs of alphoid-BACs and GCH1-BAC. CMV/a100 BAC contains 100 kb of the a21-I alphoid array from human chromosome 21 and a CMV-Bsd (Blasticidin S deaminase gene from Aspergillus terreus) selection marker for mammalian cells in the BAC vector. SV/a50 BAC contains 50 kb of the a21-I alphoid array and an SV2-Bsr (Blasticidin S deaminase gene from Bacillus cereus) selection marker. GCH1-BAC contains a 180 kb genomic DNA fragment containing the GCH1 gene. The regions used as probes for FISH analysis, Southern analysis and exons (1 to 6) of the GCH1 gene are indicated as shadowed boxes, black boxes and open boxes, respectively. BAC vectors contain chloramphenicol-resistance gene (Cm) for selection in E. coli.

FIG. 4 shows the result of FISH analysis for GCH1 signals on HAC. The cell lines HT/GCH2-10, generated by co-transfection of CMV/a100 BAC and GCH1-BAC, and the cell line HT/GCH5-18 generated by co-transfection of SV/a50 BAC and GCH1-BAC were hybridized with probes for GCH1 exon 1 (green) and BAC vector (red) (Left) or with probes for GCH1 exon 4-6 (green) and GCH1 exon 1 (red) (Right). Arrowheads indicate HACs.

FIG. 5 shows the result of structural analysis of GCH1-HAC. The result of restriction analysis of GCH1 genes in HACs is idicated. Genomic DNAs from HT/GCH2-10, HT/GCH5-18 and non-transfected HT1080 were digested with BamHI (A) or StuI (B) and fractionated by conventional gel electrophoresis. The expected size of the BamHI and StuI fragments detected by the US probe (A) and the exon 6 probe (B), respectively, using the endogenous GCH1 locus and GCH1-HAC, are shown on the top.

FIG. 6 is a graph used for estimation of the copy number of GCH1-BAC and alphoid BAC in the HACs by dot hybridization. Left: The intensity value obtained with the GCH1 exon 6 probe. Input DNA of GCH1-BAC (0.4, 0.2, 0.1 ng) and genomic DNA (1.0, 0.5 μg) from HT1080, HT/GCH2-10 and HT/GCH5-18 were hybridized with the GCH1 exon 6 probe. The value obtained with 0.1 ng GCH1-BAC DNA was used as a standard. Right: The intensity value obtained with BAC vector probe. GCH1-BAC (0.5, 0.1, 0.05 ng) and genomic DNA (0.5, 0.25 μg) from HT1080, HT/GCH2-10 and HT/GCH5-18 were hybridized with the BAC vector probe. The signal intensity obtained with each probe was determined using a Fuji image-analyzer BAS1000.

FIG. 7 shows the result of FISH analysis of hybrid cells which have been obtained by cell fusion of HAC-containing cell line with mouse A9 cells. HT/GCH5-18 cell lines were fused with A9 cells mediated by PEG BS- and Ouabain-resistant cell lines were analyzed by FISH. Metaphase spreads were hybridized with the BAC vector probe (red) and an Alu repeat probe (green) (A) or hybridized with the BAC vector probe (green) and a mouse minor satellite probe (red) (B). Arrows indicate HACs.

FIG. 8 shows the result of FISH analysis of ES cells in which HAC was transfered. A shows the result of detection using alphoid DNA and a BAC vector as probes; B shows the result of detection using an exon 1 region of GCH1 and a BAC vector as probes; and C shows the result of detection using mouse minor satellite DNA and a BAC vector as probes.

FIG. 9 is a graph showing the result of an analysis of the stability of HAC in ES cells. Black box shows the rate of HAC-containing cells in the case where culturing is carried out in the presence of blasticidin S (bs+); and void box shows the rate of HAC-containing cells in the case where culturing is carried out in the absence of blasticidin S (bs−).

FIG. 10A shows the results of PFGE analysis of A201F4.3 (lane 1 and lane 2) and 7c5hTEL (lane 3 and lane 4). In addition to a chromosome of the host cell, the presence of globin or alphoid YAC is observed at 150 kb or 100 kb (lane 1 and lane 3). Purified and condensed YACs (lane 2 and lane 4) and mixed YAC (5 lane) were introduced into HT1080 cells. M in the view indicates a molecular weight marker. FIG. 10B shows the results of FISH analysis of transformed cells obtained by the introduction of YAC. An arrow shows a mini chromosome observed in the transformed cell (upper view). Furthermore, signals of arm portions of YAC (green: arrow heads) and alphoid (red: arrow) are shown (lower view). Staining was carried out by using DAPI (blue).

FIG. 11 shows the results of FISH analysis of transformed cells containing mini chromosomes. The result in the case of using arm portions of YAC (green: arrow heads) and alphoid (red: arrow) as probes (left upper view), the result in the case of using A (green, arrow head) of β globin shown in the lower part of FIG. 11 and alphoid (red, arrow) (upper right view), the result in the case of using B (green, arrow head) of β globin and alphoid (red, arrow) (lower left view), and the result in the case of using C (green, arrow head) and alphoid (red, arrow) (lower right view) are shown, respectively.

FIG. 12 shows the results of FISH analysis of two clones (C11 and C29) that are transformed cells containing a mini chromosome by using a human β globin (SEQ ID NO. 5, SEQ ID NO. 6, and SEQ ID NO. 9) or telomere repeat sequence (about 500 bp of sequence consisting of sequences of SEQ ID NO. 8) as probes. Blue, green and red indicate signals of DAPI, human β globin and telomere, respectively.

FIG. 13 shows the results of FISH analysis of transformed cells obtained by fusing A9 cells and cells containing a mini chromosome. The upper left view shows the result of staining with DAPI (blue), the upper right view shows the result of detection of signal (green) by β globin probe (SEQ ID NO. 5, SEQ ID NO. 6 and SEQ ID NO. 9), the lower left view shows the result of detection of signal (red) by an alphoid probe (SEQ ID NO. 3) and the lower right view was obtained by superposing the above-mentioned views. An alphoid signal can be observed only in the mini chromosome.

FIG. 14 shows the results of fiber FISH analysis of a mini chromosome. The upper view shows the result when a β globin probe (SEQ ID NO. 5, SEQ ID NO. 6, and SEQ ID NO. 9) was used, the middle view shows the result when an alphoid probe (SEQ ID NO. 3) was used, and the lower view was obtained by superposing the above-mentioned two results. Signals of alphoid and B globin are represented by red and green, respectively.

FIG. 15 shows the result of analysis of transcription amount of globin gene in HAC-containing cells. The upper part shows the results of analysis by a RT-PCR, and the lower part shows the results of analysis by a real-time PCR.

FIG. 16(a) shows a chimeric mouse created by using HAC-containing ES cell lines. FIG. 16(b) shows the results of PCR analysis of DNA derived from various organs of a child mouse (24 hours after its birth) obtained by natural childbirth from a mouse (provisional parent) transplanted with an embryo into which HAC-containing ES cells are introduced. TT2 indicates an ES cell, TT2/GCH2-10 indicates HAC-containing ES cell, brain indicates the brain, heart indicates the heart, thymus indicates the thymus, liver indicates the liver, spleen indicates the spleen, and kidney indicates the kidney, respectively. Furthermore, c1 to c15 indicate individual bodies, respectively. FIG. 16(c) shows the results of FISH analysis of a mouse individual body created by using ES cells. Signals of the alphoid array and signals of BAC vector are observed (arrow head).

FIG. 17 shows a chimeric mouse created by using XO nuclear type ES cell lines containing HAC.

FIG. 18 shows a characterized portion of an acceptor precursor BAC-LCR-lox71 used for construction of a mammalian artificial chromosome.

FIG. 19 shows the results of measurement of EGFP intensity in an artificial chromosome constructed by using a precursor including human 13 globin LCR and a lox site. HAC: artificial chromosome constructed by using a precursor including human β globin LCR and a lox site, INT1 and INT2: two cell lines with highest two fluorescence intensities selected from stable cell lines into which pEGFP-C1 is introduced on random places of the chromosome. The lower right graph summarizes measurement results.

### BEST MODE FOR CARRYING OUT THE INVENTION

The first aspect of the present invention relates to a production method of a mammalian artificial chromosome and includes a method using a circular vector as a precursor and a method using a linear vector as a precursor. Note here that a mammalian artificial chromosome is also referred to as MAC and this includes a human artificial chromosome (hereinafter, which is also referred to as “HAC”).

(Vector as Mammalian Artificial Chromosome Precursor)

In the present invention, as a mammalian artificial chromosome (MAC) precursor, a first vector (circular vector or yeast artificial chromosome) and a second vector (circular vector or yeast artificial chromosome) are used. The first vector includes a mammalian centromere sequence and supplies centromere necessary for replication and maintaining of MAC. On the other hand, the second vector includes a functional sequence and becomes a source of a functional sequence incorporated into the MAC. It is possible to use plural kinds of second vectors including different functional sequences therein. That is to say, for example, MAC of the present invention can be produced by using, for example, a first vector and two kinds of vectors including different functional sequences therein. In this way, when plural kinds of second vectors are used, it is possible to construct a MAC that holds a plurality of functional sequences in a state of being capable of expressing. This signifies that the MAC of the present invention can be used as, for example, a tool for introducing a plurality of genes which are acting cooperatively.

As the first vector and second vector, circular vector or linear vector can be used. As the circular vector, a BAC (bacterial artificial chromosome) or a PAC (P1 artificial chromosome) capable of autonomously replicating in bacteria (for example, E. coli) can be used. It is advantageous to use BAC or PAC in that introducing operation, amplification and maintaining, etc. are easy and various kinds thereof are available.

The circular vector used in the present invention can be constructed by providing necessary modification for a known BAC or PAC. For example, Belo-BAC (New England Biolabs inc., Beverly, Mass. 01915-5599) is used as the starting material, and an insertion site for a mammalian centromere sequence is produced therein by restriction enzyme treatment, etc., followed by inserting a mammalian centromere sequence, which has separately been prepared, into this insertion site. Thereby, the circular vector (first vector) including a mammalian centromere sequence can be constructed. On the other hand, the vector (second vector) including a functional sequence can be prepared from a library if a library including the clone thereof is provided. Needless to say, similar to the first vector, the second vector also may be produced from a known vector by genetic engineering technique.

As the linear vector, a DNA construct (yeast artificial chromosome, hereinafter, which is also referred to as “YAC”) that functions as a chromosome in yeast is used. The first vector in this case includes at least a mammalian centromere sequence and a telomere sequence. Herein, “mammalian telomere” denotes a repeat sequence existing in the telomere region of a chromosome in mammalians. Human telomere is composed of repeated 5′-TTAGGG-3′. It is preferable to use a centromere sequence including the repetition of this sequence when a human artificial chromosome (HAC) is produced.

It is preferable that the first vector and/or the second vector include a selection marker gene. It is advantageous because when the transformation (transfection) is carried out by using these vectors, transformed cells can be selected easily by using the selection maker gene. It is preferable that only one of the vectors includes a selection marker gene. It is advantageous because by reducing the number of selecting makers to be used, selection operations necessary for the process of the production of a MAC or the use thereof can be simplified.

Furthermore, it is preferable that only the first vector includes a selection maker gene. According to such a configuration, by using the selection marker gene, it is possible to select transformed cells into which mammalian centromere sequences are appropriately introduced. In other words, it is possible to effectively select transformed cells with high possibility of containing DNA contracts that function as a chromosome. On the other hand, since it is not necessary to insert a selection marker into a vector (second vector) including a functional sequence, advantageously, intact vectors prepared from a commercially available library consisting of clones without including selection marker genes are used (i.e., without carrying out the insertion of the selection marker gene) as the second vector. In addition, since the second vector need not include a selection marker gene, the insert DNA to be inserted into the second vector has room by the size of the selection marker gene. As a result, it is possible to construct a MAC containing a larger sized functional sequence.

(Mammalian Centromere Sequence)

In the present invention, “mammalian centromere sequence” denotes a sequence that functions as a centromere in mammalian cells. As the mammalian centromere sequence, for example, a sequence derived from an alpha satellite region of a human chromosome can be used. Herein, “a sequence derived from an alpha satellite region” denotes a part or entire of the alpha satellite region or a sequence obtained by partially modifying any of the sequences. Herein, “partially modifying” denotes substitution, deletion, insertion and/or addition of one or plurality of bases in the sequence of interest. Such modification may be given to a plurality of regions.

In the alpha satellite region of a human chromosome, in general, a plurality of sequences referred to as a CENP-B box consisting of 5′-NTTCGNNNNANNCGGGN-3′ (SEQ ID NO: 1) are disposed at regular intervals (Masumoto et al. NATO ASI Series. vol. H72, Springer-Verlag. pp 31-43, 1993; Yoda et al. Mol. Cell. Biol., 16, 5169-5177, 1996). The mammalian centromere sequence of the present invention preferably includes a region having this CENP-B box with high frequency.

It is preferably to use a sequence derived from an alpha satellite region of a human chromosome 21. The alpha satellite region of the human chromosome 21 has been investigated in detail and has a region called α21-I. The α21-I region includes a sequence called an alphoid 11mer repeat unit. This repeat unit includes a plurality of CENP-B boxes consisting of 5′-NTTCGTTGGAAACGGGA-3′ (SEQ ID NO: 2) at regular intervals (Ikeno et al. Human Mol. Genet., 3, 1245-1247, 1994).

Preferably, the mammalian centromere sequence of the present invention includes a plurality of such alphoid 11mer repeat units. A sequence isolated from the alphoid region of the human chromosome 21 so as to be identified is shown by SEQ ID NO: 3 (about 25 kb alphoid fragment).

The centromere sequence has a sufficient length to form a centromere having an appropriate function in the constructed mammalian artificial chromosome. For example, a centromere sequence having a size of about 25 kb to about 150 kb (for example, about 50 kb, about 80 kb and about 100 kb) is used. A centromere sequence having size of preferably about 80 kb or less and further preferably about 50 kb or less is used. The use of a small-sized centromere sequence facilitates operations such as separation, purification of the first vector including the centromere sequence, and furthermore reduces the probability of exfoliation and modification, which possibly occur at the time of cloning and/or proliferation. Herein, as shown in Examples mentioned later, in an example in which a circular vector (BAC) was used, even in the case where about 50 kb alphoid DNA was used as a centromere sequence, it was confirmed that an artificial chromosome capable of appropriately forming a centromere/kinetochore structure was constructed. Similarly, in an example in which a linear vector (yeast artificial chromosome) was used, even in the case where about 80 kb alphoid DNA was used as a centromere sequence, it was confirmed that an artificial chromosome capable of appropriately forming a centromere/kinetochore structure was constructed.

The mammalian centromere sequence can be prepared from an appropriate human cell, fusion cell containing human chromosome such as WAV17, or non-human mammalian cells. For example, one of these cells is fixed as an agarose plug, followed by purifying and condensing DNA fragments including the target centromere sequence by way of restriction enzyme treatment, pulsed-field gel electrophoresis (hereinafter, referred to as “PEGE”) and the like. Then, the DNA fragments are cloned to an appropriate vector and stored before use.

On the other hand, when the library including a clone containing a mammalian centromere sequence is available, it is possible to obtain a mammalian centromere sequence appropriately from the library by way of restriction treatment. For example, a21-I alphoid fragment is obtained by using the LL21NC02 library (Lawrence Livermore Laboratory) and this fragment can be used as a mammalian centromere sequence. A mammalian centromere sequence may be constructed by using a plurality of the obtained α21-I alphoid fragments. Furthermore, a plurality of α21-I alphoid fragments which differ in size from each other are obtained and by combining these fragments, a mammalian centromere sequence may be constructed.

(Mammalian Replication Origin)

In general, a mammalian centromere sequence has one or more replication origins. Therefore, usually, the first vector including a mammalian centromere sequence includes a mammalian replication origin. In the case where the mammalian centromere sequence does not include mammalian replication origin, the first vector or the second vector is allowed to include a mammalian replication origin additionally. However, this is not required when the functional sequence contained by the second vector has already include a mammalian replication origin.

(Functional Sequence)

The functional sequence is a sequence capable of exhibiting specific effects by the expression thereof and typically consists of a sequence encoding the target gene and the regulatory region thereof. As the functional sequence of the present invention, a sequence having a function of suppressing the expression of a certain gene and suppressing the activity of a certain RNA upon expression thereof, and the like, for example, a sequence encoding a so-called antisense RNA or ribozyme RNA, etc., can be used.

As the target gene, various genes can be employed and examples thereof may include a human guanosine triphosphate cyclohydrolase I (GCH1) gene, human β globin gene cluster, a tumor suppressor gene such as RB and p53, an apoptosis induction gene such as c-myc and p53, genes encoding cytokine, various growth factors, antibody, tumor antigen, etc. and the like. A sequence encoding the target gene may be genome DNA or cDNA.

As the functional sequence, it is possible to use a sequence encoding a plurality of target genes. As such a sequence, a sequence including a base sequence corresponding to a plurality of proteins in a case where the plurality of proteins are interacting with each other so as to obtain a specific effect, and a sequence including a base sequence corresponding to a plurality of enzymes necessary for a series of reaction system. In such cases, it is possible to use a sequence for controlling the expression for each sequence corresponding to each expression product. However, a sequence capable of controlling the expression of all or a part (two or more) expression product as a whole may be used. For example, a construct configured by disposing sequences corresponding to a plurality of expression products under the control of one promoter sequence may be used.

Sequence of the target gene can be prepared by, for example, a known library. In a case where a library consisting of vector clones including a sequence of the target gene (and the regulatory region thereof) is available, a vector containing a sequence of the target gene (and regulatory region thereof) prepared from the library can be used as the second vector (or production material thereof). For example, BAC libraries such as CITB (California Institute of Technology) Human BAC Libraries, RPCI-11 (Roswell Park Cancer Institute) Human BAC Library (Keio University), CITB Mouse BAC Library, RPCI-22 Mouse BAC Library, etc., PAC libraries such as RPCI Human PAC Libraries, RPCI-21 Mouse PAC Library, etc., or YAC libraries such as CEPH Human YAC Library, Washington University Human YAC library, WI/MIT 820 YAC Library, Whitehead I Mouse YAC Library, etc. (which are provided by Reseach Genetics, 2130 Memorial Parkway SW, Huntsville, Ala. 35801, US) can be used.

In the present invention, since a vector with large cloning capacity is used, a large-sized DNA fragment including a regulatory region in addition to the structural gene can be used as a functional sequence. In principle, the regulatory region herein means the regulatory sequence of a the target gene (a sequence of the region directly involved in the regulation of the target gene in the chromosome), however, it may include a sequence in which partial modification is provided to this as long as the function is maintained. “Partial modification” herein denotes substitution, deletion, insertion and/or addition of one or plurality of bases in the sequence of interest. Such modifications may be done to the plurality of regions.

It is possible to use a second vector including a sequence for specifically inserting a sequence of interest (in the present invention, which is referred to as “inserting sequence”) as a functional sequence. By using such a second vector, it is possible to construct a general-purpose mammalian artificial chromosome (MAC) to which a predetermined sequence can be inserted later. The sequence of interest herein denotes typically a sequence encoding genes of interest (preferably, a sequence including a sequence encoding the regulatory region together). However, the sequence is not particularly limited thereto and may be a sequence having a function of suppressing a predetermined gene or a function of suppressing a predetermined RNA, and the like. For example, the sequence may be a sequence encoding a so-called antisense RNA or a ribozyme RNA, etc.

The kinds of the inserting sequences are not particularly limited, but loxP site or FRT (Flp Recombination Target) site can be preferably used. For example, when the loxP site is used, firstly, a MAC having the loxP site is produced and Cre recombinase is allowed to act on this, whereby a sequence of interest can be introduced site-specifically and finally a MAC including the sequence of interest can be constructed. Similarly, when a MAC having an FRT site is produced, Flp ricombinase is used so as to finally construct a MAC including a sequence of interest. Note here that even a sequence obtained by modifying a part of the lsxP site or the FRT site, etc. can be used as an inserting sequence as long as it has a function for inserting a sequence of interest. Examples of modification include deletion, addition or substitution of a part thereof, thereby increasing the introduction efficiency or enabling only introduction reaction to be carried out specifically.

By adjusting the ratio of the first vector including a mammalian centromere sequence and the second vector including the inserting sequence as a functional sequence, it is possible to change the number of inserting sequences incorporated into a mammalian artificial chromosome to be produced. Furthermore, when the mammalian artificial chromosome is produced by the co-introduction of such first vector and second vector, it is possible to incorporate the inserting sequence at a distance from a centromere (i.e. location which is not between centromere) in a mammalian artificial chromosome to be produced, so that a mammalian artificial chromosome that holds an insertion sequence functioning appropriately can be constructed.

It is preferable that the second vector to be used in the present invention has an insulator sequence. Herein, the insulator sequence is a base sequence characterized by exhibiting an enhancer blocking effect (expressions of neighboring genes are not affected by each other) or a chromosome boundary effect (a region assuring the gene expression and a region suppressing the gene expression are separated with each other). It is expected that the use of the insulator sequence promotes the expression of a target gene contained by a mammalian artificial chromosome. On the other hand, as shown in Examples mentioned below, when the above-mentioned inserting sequence such as loxP, etc. is used, if the insulator sequence is used together, it was found that the introduction rate of the target gene into the mammalian artificial chromosome was increased. Thus, when the insulator sequence is used, the effect of increasing the rate of introducing genes into the mammalian artificial chromosome can be exhibited. Therefore, it is possible to construct effectively and more certainly the mammalian artificial chromosome that holds the target gene. Usable insulator sequences are not particularly limited. It is possible to use not only an insulator, which has been identified as an insulator, but also a sequence obtained by providing modification for the sequence as long as the expected effect (the increase in promoting the expression of target gene or the increase in the gene introduction efficiency) is not reduced. A plurality of insulator sequences may be used together. When a plurality of insulator sequences are used one kind of insulator sequence may be used or plural kinds of insulator sequences in combination may be used. Note here that human B globin HS1 to 5, chicken β-globin HS4, Drosophila gypsy retrotransposon, sea urchin 5′ flanking region of arylsulfatase, blocking element α/d of human T-cell receptor α/d, repeat organizer of Xenopus 40S ribosomal RNA gene, and the like, have been known as insulator sequence.

Concrete examples of the mammalian artificial chromosome precursor (second vector) used in the case where the insulator sequence is used include one having an inserting sequence of loxP etc. as a functional sequence and having an insulator sequence at the 5′ side of the inserting sequence can be used.

In the mammalian artificial chromosome precursor (second vector), an insulator sequence may be disposed at 3′ side instead of 5′ site of the inserting sequence. Alternatively, a mammalian artificial chromosome precursor (second vector) in which insulator sequences are disposed at both sides so that they sandwich the inserting sequence. Furthermore, when an insulator sequence is disposed at any positions, a plurality of insulator sequences may be continuously disposed or may be disposed with other sequence interposed therebetween.

(Host Cell)

As a host cell into which the first vector and the second vector are introduced, a host cell in which the recombination of the both vectors is carried out can be used. For example, human fibroblast cell line such as HT1080 cells, HeLa cells, CHO cells, K-562 cells, and the like may be used as a host cell.

(Production Method for Mammalian Chromosome)

The production method of the mammalian artificial chromosome (MAC) of the present invention includes (1) a first step of introducing a first vector including a mammalian centromere sequence and a second vector including a functional sequence into a mammalian host cell; (2) a second step of selecting transformed cells; and (3) a third step of selecting a cell containing a MAC from the selected transformed cells.

The method of introducing the first vector and the second vector in the first step is not particularly limited. However, it is preferable that these two vectors are introduced into the mammalian host cell at the same time. It is advantageous because recombination between the vectors in the mammalian host cell is carried out efficiently. It is also advantageous because introduction operation can be simplified. For introducing two vectors at the same time, for example, firstly both vectors, which were mixed with each other prior to the introduction operation, may be introduced into the host cell.

The amount ratio of the first vector and the second vector to be introduced is, for example, first vector: second vector=about 10:1 to about 1:10 in a molecular ratio so that a MAC containing a functional sequence in a state capable of expressing is appropriately formed. Preferably, the ratio is first vector:second vector=about 1:1. Herein, when the amount of the first vector is too small, a MAC including active centromere may not be formed. Meanwhile, when the amount of the second vector is too small, a functional sequence may not be taken into a MAC. On the other hand, the increase in the amount of the second vector enables efficient taking of the functional sequences. As a result, the construction of the MAC including plural copies of the functional sequences can be expected. As shown in the following example, according to the production method of the present invention, the construction of mammalian artificial chromosomes containing plural copies of a target gene has been achieved. In the MAC including plural copies of a target gene, the total amount of expression of the target genes is necessarily increased. Therefore, in the case where the MAC of the present invention is used as a vector for introduction the target genes, high expression efficiency in the cell, in which the MAC has been introduced, can be obtained. This is particularly useful in the case where the MAC of the present invention is used as a vector for gene therapy. This is also beneficial in the case where the MAC of the present invention is used as a material for evaluating the operation/effect of drugs or candidate compounds of drugs.

The method of introducing each vector into the host cell is not particularly limited. Methods such as lipofection (Felgner, P. L. et al., Proc. Natl. Acad. Sci. U.S.A. 84, 7413-7417(1984)), transfection using calcium phosphate, microinjection (Graessmann, M. & Graessmann, A., Proc. Natl. Acad. Sci. U.S.A. 73, 366-370(1976)), electroporation (Potter, H. et al., Proc. Natl. Acad. Sci. U.S.A. 81, 7161-7165(1984)), and the like can be employed.

In the host cell, the recombination between the first vector and the second vector occurs. As a result, a MAC including a centromere sequence derived from the first vector and a functional sequence derived from the second vector can be formed.

After the first vector and the second vector are introduced, transformed cells (transformants) are selected (second step). The selection of the transformed cells can be carried out by selectively culturing the cells after introduction of the vectors by using the selection marker gene which was inserted in the first vector or second vector in advance. Note here that as a result of isolating cells arbitrarily from the cell group to which both vectors were introduced, the isolation operation in the case where the isolated cells are transformed cells is encompassed in the “selection of transformed cells” according to the present invention.

After the transformed cells are selected, a cell containing a MAC is selected (third step). Such a selection operation can be carried out by a detection method using a probe or antibody specific to MAC. Concretely, for example, it can be carried out by in situ hybridization method using a probe that hybridizes specifically with respect to at least a part of the mammalian centromere sequence included in the first vector. In this step, in order to confirm that a MAC is formed in which the second vector is appropriately incorporated, it is preferable to carry out the similar hybridization analysis using a probe that specifically hybridizes at least a part sequence (for example, functional sequence) specific to the second vector. For detection of each probe used in the above mention, fluorescent substance, radioactive substance, etc. can be used. A method of using a fluorescent substance as a label of the probe is referred to as FISH (Fluorescence in situ hybridization) method and enables safe and simple detection of MAC (Lawrence, J. B. et al. Cell 52:51-61, 1998; Takahashi, E. et al. Jpn. J. Hum. Genet. 34:307-311, 1989).

It is preferable to carry out a step of confirming that MAC in which a functional sequence is appropriately incorporated is formed in addition to the third step. Such a confirming step can be carried out, for example, by detecting the expressed product of the gene in a case where the functional sequence includes the target gene.

The mammalian artificial chromosome (MAC) obtained by the above-mentioned production method can be maintained extremely stably even under the non-selective conditions. Note here that “non-selective condition” means a condition that dose not include the selective operation enabling the existence of only cells in which a MAC is present.

Although it may be different depending upon the kinds of precursor vectors and host cell to be used, etc., according to the production method of the present invention, it is possible to allow about 95% or more of cells (group) to hold a MAC after about 30 days (after about 30 passages) under non-selective conditions after DNA construct (first vector and second vector) is introduced into the host cell. Furthermore, it is possible to maintain the state that one copy of MAC is present in the cell (see Example mentioned below).

It is preferable that the number of MACs contained by the finally obtained transformed cells (mammalian cells) is fewer, and it is particularly preferable that one MAC per nucleus is contained. According to the production method of the present invention, it is possible to efficiently obtain transformed cells containing one mammalian artificial chromosome per nucleus.

Another aspect of the present invention is to provide a transformed cell (transformant) containing a mammalian artificial chromosome (MAC) produced by the above-mentioned method. Such a transformed cell can be used as a supply source for transferring MACs to the other cells. Furthermore, such a transformed cell can be used as a carrier for introducing a mammalian artificial chromosome into the living body by, for example, introducing the transformed cell per se into the living body.

(Properties of Mammalian Artificial Chromosome)

The mammalian artificial chromosome (MAC) constructed in the present invention is characterized by (1) having a mammalian replication origin, a mammalian centromere sequence, and a functional sequence (a sequence encoding a target gene and the regulatory region thereof or an inserting sequence for inserting a sequence of interest); (2) being replicated in mammalian cells; (3) being maintained extrachromosomally in a host cell; (4) being transmitted to daughter cells at the time of cell division; and (5) being circular or linear in form. In a case where the MAC is produced by using a circular vector (BAC or PAC) as a precursor, its form becomes circular because a telomere sequence is not included. On the other hand, in a case where the MAC is produced by using a linear vector (yeast artificial chromosome) as a precursor, it is thought that when telomere sequences that function sufficiently are provided at the both ends, the MAC has a linear form and that if not so, the MAC has a circular form. Note here that the mammalian replication origin may exist in a mammalian centromere sequence.

According to the above-mentioned characteristics, the MAC of the present invention functions as a chromosome in a mammalian cell into which a MAC is introduced and is appropriately segregated to daughter cells so as to be maintained without accompanying substantial change of the structure at the time of cell division.

Furthermore, in the MAC of the present invention, the target gene of interest can be maintained together with its regulatory region and allowed to express the target gene sufficiently in the cell into which the MAC is introduced. Note here that as shown in Examples mentioned below, in an example in which GCH1 gene was used as the target gene, wee realized regulation of expression that is same as in the case existing on the chromosome.

The mammalian artificial chromosome of the present invention may include a DNA sequence which enables the mammalian artificial chromosome to autonomously replicate and being segregated in cells other than mammalian cells (for example, yeast cells, bacteria such as E. coli). Since such a DNA sequence is included, the MAC of the present invention can function as a chromosome also in cells other than mammalian cells. Therefore, the MAC of the present invention can be used as a shuttle vector.

It is preferable that a mammalian centromere sequence include a CENP-B box sequence. It is particularly preferable that a region expressing CENP-B boxes with high frequency is included. Furthermore, it is preferable that the mammalian centromere sequence includes a sequence derived from alpha satellite region of the human chromosome 21, and particularly a sequence of α21-I alphoid region.

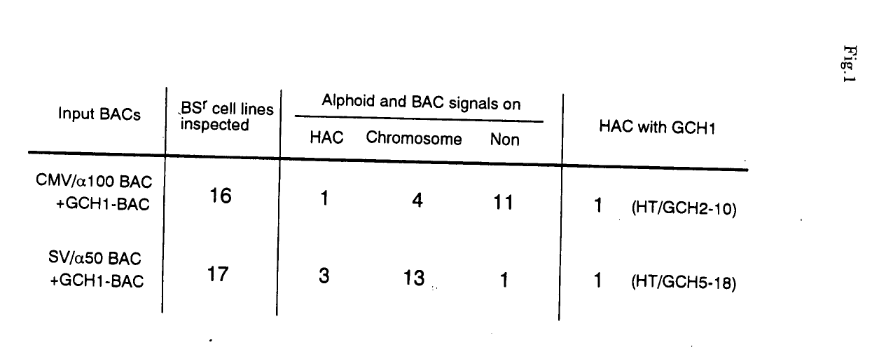

As shown in Examples mentioned later, the present inventors succeeded in production of a human artificial chromosome (HAC) containing about 180 kb gene encoding human GCH1 (EC 3.5.4.16; GCH1) in a state capable of expressing in a system using a BAC as a precursor. One human GCH1 gene is located in the chromosome 14q22.1-q22.2 and the gene is composed of six exons spanning more than 60 kb (FIG. 1) (Ichinose et al. 1995; Hubbard et al. 2001). GCH1 is the first enzyme for the biosynthetic pathway of tetrahydrobiopterin, the essential cofactor for enzymatic reactions as described below and is present in higher organisms (Nichol et al. 1985; Tanaka et al. 1989; Werner et al. 1990). Tetrahydrobiopterin is synthesized from GTP in a three-step reaction by GCH1,6-pyruvoyl-tetrahydropterin synthase (EC 4.6.1.10; PTPS) and sepiapterin reductase (EC 1.1.1.153; SR). Among these enzymes, the major controlling point is GCH1, the expression of which is under the control of cytokine induction (Werner et al. 1993) and the feedback regulatory protein, GFRP, at the transcriptional and post-translational levels, respectively. Tetrahydrobiopterin functions as a natural cofactor of the aromatic amino acid hydroxylases; phenylalanine hydroxylase (EC 1.14.16.2; PAH), tyrosine hydroxylase (EC 1.14.16.3; TH), the first and rate-limiting enzyme of dopamine synthesis, tryptophan 5-hydroxylase (EC 1.14.16.4; TPH), serotonin biosynthesis. Tetrahydrobiopterin is also essential for all three forms of nitric oxide synthase (NOS) (Kaufman 1993). Decreases in GCH1 activity, tetrahydrobiopterin level and/or TH activity causes dopamine deficiency in the nigrostriatum dopamine neurons and provokes several well-known clinical symptoms, such as hereditary dopa-responsive dystonia (DRD/Segawa's syndrome) (Ichinose et al. 1994) or parkinsonism. Thus, HACs carrying the GCH1 gene with the authentic regulatory region would almost certainly prove useful for compensating for the defects in the GCH1 gene as well as facilitating a close study of the complex regulatory mechanism of GCH1 gene expression in vivo.

(Transfer of Mammalian Artificial Chromosome)

The introduction of a mammalian artificial chromosome (MAC) into a mammalian cell can be carried out by, for example, the following method.

First of all, from a host cell containing a MAC, the MAC is isolated. The isolated MAC is introduced into a mammalian cell (target cell). The isolation of MAC can be carried out by, for example, the following method. First of all, suspension of the host cells containing the MAC is prepared and a nucleic acid component is extracted. Thereafter, fractions containing a chromosome is obtained by density-gradient centrifugation using Ficoll, etc. Then, artificial chromosomes with small molecular weight are separated by using a filter, etc.

An example of the method of introducing the separated MAC into mammalian cells includes lipofection, transfection using calcium phosphate, microinjection, electroporation, and the like.

A MAC can be introduced into mammalian cells by the following method using cell infusion. First of all, host cells containing a MAC and mammalian cells capable of forming micronuclei are fused to each other, followed by selecting hybrid cells which are capable of forming micronuclei and hold MAC from the fused cells. Herein, as the mammalian cells capable of forming micronuclei, for example, A9 cells (American Type Culture Collection, Manassas, Va. 20110-2209), mouse ES cells, CHO cells, and the like can be used. The cell infusion can be carried out by using PEG (Polyethlene Glycol). The selection of the target hybrid cells can be carried out by a selection culture using a selection marker specific to the host cell used in the cell infusion and ouabain in the case where, for example, mouse A9 is used.

Then, micronuclei are formed from the selected hybrid cells. In general, micronucleate multinuclear-cells are formed by colcemid treatment, followed by carrying out cytochalasin B treatment and centrifugation so as to micro-cells.

The micro-cells are fused to mammalian cells (target cells) by fusion using PEG, etc. From the above-mentioned step, MACs are transferred (introduced) to mammalian cells, so that mammalian cells containing the MAC can be obtained.

Herein, example of the target cells include cells forming a certain tissue of human or non-human mammalian (mouse, rat, etc.) (fibroblast cells, endothelial cells, cardiac muscle cells), germ cells (including a fertilized egg), embryonic stem cells (ES cells), embryonic germ cells (EG cells), tissue stem cells (hematopoietic stem cells, mesenchymal cells, nervous system stem cells, osseous system stem cells, cartilage stem cells, epithelial stem cells, hepatic stem cell, etc.), and the like. Cells obtained by providing such stem cells with induction treatment for allowing them to differentiate into cells of specific tissue can be used as the target cells. Examples of such target cells include cells obtained by differentiated-inducing nervous system stem cells to neuron, astrocyte and oligodendrocyte by using a platelet-derived growth factor (PDGF), a ciliary derived neurotrophic factor (DNTF) and triiodothyronine (T3), respectively; cells obtained by differentiated-inducing mesenchymal cells to osteoblast by using dexamethasone and ascorbic acid, and the like; and cells obtained by differentiated-induciong mesenchymal cells to cartridge cells by culturing in the presence of TGF-β, etc.

As the cells into which the MAC of the present invention is introduced, cells of vertebrate animal other than mammalian, for example, Pisces (Aplocheilus latipes, zebrafish, etc.), Amphibia (Xenopus laevis, etc.), Aves (chicken, quail, etc.), and the like may be used.

The transferring of the MAC into the target cells is carried out in vitro, in vivo or ex vivo. For example, by directly transferring a mammalian artificial chromosome (MAC) into the cells in vivo, or by introducing cells into which a MAC is transferred ex vivo into a living body, the MAC can be introduced into the site of interest (for example, specific tissue such as heart, lungs, etc.). As a result, expression is carried out from a functional sequence contained in the MAC in the introduction site. In this way, a MAC can be used as a vector for introducing a foreign gene into the living body. Since a MAC has a large cloning capacity, in particular, it can be preferably used as a vector for introducing a large foreign gene including a regulatory region.

More concretely, the mammalian artificial chromosome (MAC) of the present invention can be used as a vector for, for example, gene therapy. That is to say, the MAC of the present invention can be used for the introduction of foreign genes for the purpose of compensating the function of defective genes, suppression of expression of abnormal genes, or suppression of the effect of the expressed products. Since the MAC of the present invention can be maintained stably in the cell into which the MAC is introduced, the transgene is expressed stably and for a long term. Thus, excellent therapy effect can be expected. Furthermore, since a large-sized foreign gene including regulatory region can be introduced when the MAC of the present invention is used, gene expression under the control of original regulatory region can be carried out. Also from this viewpoint, excellent therapy effect can be expected.

Furthermore, the MAC of the present invention also provides a means for clarifying the function or the action mechanism of the gene of interest. In particular, it is useful to provide a means for clarifying the function or action mechanism of a gene, which was not able to be introduced by a conventional vector due to it large size. That is to say, it provides a means for studying of a gene whose function or action mechanism is unknown. In particular, since the MAC of the present invention can hold foreign genes so that they can express under the control of the original regulatory region, analysis of tissue specific expression mechanism or analysis of expression of a human gene which has been introduced into a model animal individual body such as a mouse, and the development of inhibitors and promoters.

As shown in Examples mentioned later, the present inventors succeeded in creating a mouse (chimeric mouse) into which the mammalian artificial chromosome (MAC) of the present invention is introduced by using ES cell. Note here that the present inventors succeeded in not only the creation of chimeric mouse (male) using XY nuclear type ES cells but also the creation of chimeric mouse (female) using XO nuclear type ES cells. Thus, it was confirmed that the mammalian artificial chromosome of the present invention could be used for creating transformed animals. Based on such results, another aspect of the present invention provides non-human transformed animal in which a mammalian artificial chromosome is introduced and the method for creating the same. Examples of the non-human transformed animals include Rodent such as mouse, rat, and the like, but not limited thereto.

The non-human transformed animals can be created by introducing the MAC at its development stage. As the creating method, a method using ES cells, a microinjection method in which introduction of nucleus construct (MAC) is directly infused to the pronucleus of fertilized egg, and the like, can be employed. Hereinafter, as a concrete example of the method of creating the non-human transformed animals of the present invention, a method using mouse ES cells will be described. In this method, first of all, ES cells containing a MAC are prepared. Such ES cells can be prepared by using the above-mentioned micronucleus fusion method. That is to say, first of all, cells containing a MAC having a desired configuration (for example, HT1080) are prepared and fused to cells having the ability of fusing micronuclei (for example, mouse A9 cells) so as to transfer the MAC. Thereafter, micronucleus is formed by, for example, colcemid treatment from cells into which the MAC is appropriately transferred. The obtained micronucleus is fused to ES cells by, for example, use of PEG, and the like. Then, from the fused cells, one containing the MAC is selected. The thus prepared ES cells containing the MAC are introduced into the blastocyst of mouse. That is to say, first of all, after the entire uterus including the ovary is extracted from the mated female mouse, the blastocyst is collected from the uterus, and ES cells containing a HAC is introduced into the blastocyst cavity of the blastocyst by microinjection. Then, the blastocyst which the injection was completed is transplanted into the uterus pseudopregnancy mouse (provisional parent) so as to obtain a child mouse (fetus) by natural childbirth or Cesarean section.

Note here that it can be confirmed that the MAC is introduced into the obtained child mouse by observation of hair color of the child mouse or DNA analysis using a probe having a sequence specific to the used MAC.

### EXAMPLE 1

### Construction of Alphoid-BAC

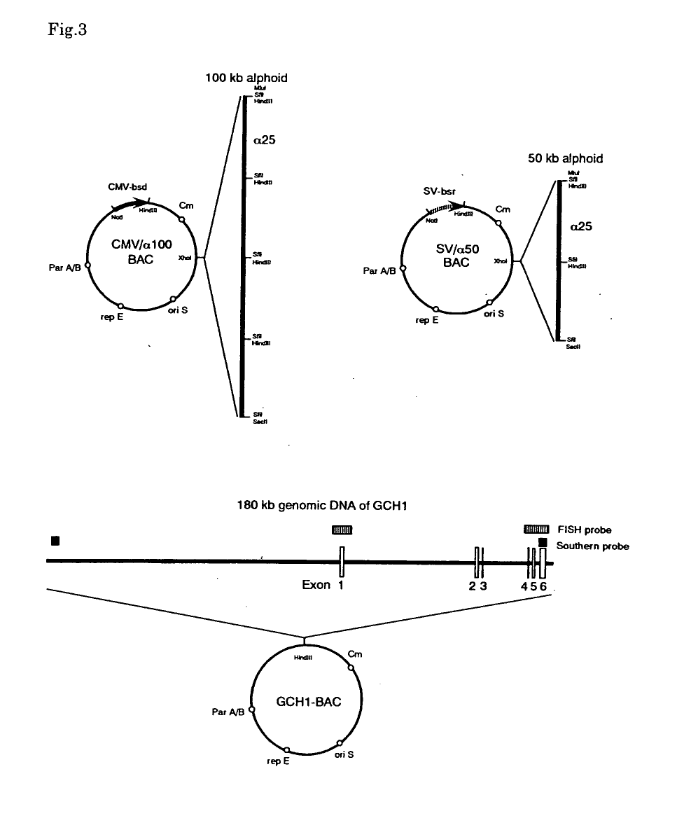

pBAC-TAN was created by insertion of a MluI-SfiI-SacII linker into the XhoI site of Belo-BAC. pBAC-CMV and pBAC-SV were created by insertion of a 1.3 kb NotI-HindIII fragment from pCMV/Bsd (Invitrogen) or a 2.6 kb PvuII-EcoRI fragment from pSV2bsr (Kakenseiyaku), both contain a Blasticidin S resistance gene, into the NotI-HindIII sites of pBAC-TAN. The 25 kb alpha 21-I alphoid fragment (α25: SEQ ID No: 3) was isolated from the cosmid clone, Q25F12, obtained from the LL21NC02 library (Lawrence Livermore Laboratory) by SfiI digestion and cloned into the SfiI site of pBAC-TAN. The resulting alphoid-BACs which contain either 50 kb or 100 kb of tandem alphoid insert were digested with MluI and SacII, and the alphoid fragments were inserted into the MluI-SacII sites of pBAC-CMV or pBAC-SV, respectively. As a result, SV/α50 and CMV/α100, which are alphoid-BACs containing 50 kb (SV/α50) and 100 kb (CMV/α100) alphoid fragments, were obtained (FIG. 3).

### EXAMPLE 2

### Generation of HAC Containing the GCH1 Genomic Locus

Alpha 21-I alphoid, consisting of an 11mer higher order repeat unit derived from human chromosome 21 (Ikeno et al. 1994), is able to generate a HAC efficiently when introduced into HT1080 cells (Ikeno et al. 1998). We generated HACs containing a GCH1 genomic locus with naturally regulated gene expression, utilizing alphoid-BACs and GCH1-BAC. BACs used in this study are shown in FIG. 3. CMV/a100 contains 100 kb of an a21-I alphoid array and a CMV-Bsd as a selectable marker, and SV/a50 contains 50 kb of an α21-I alphoid array and a SV2-Bsr selection marker. The GCH1-BAC was obtained from a BAC library (Genome systems) and has a 180 kb genomic DNA fragment containing the GCH1 gene. BAC-DNAs were purified by CsCl banding using a gradient.

We co-transfected either one of the alphoid-BACs and the GCH1-BAC in a 1:1 molecular ratio into HT1080 cells by lipofection and isolated Blasticidin S (BS) resistant cell lines after 10 days. Specifically, for generation of HAC, 0.5 μg of alphoid-BACs and 1.0 μg of GCH1-BAC (186L09, Genomesystems) were co-transfected into HT1080 cells (5×10⁵) using lipofectamine (Gibco BRL) according to the manufacturer's instructions. The cells were selected with 4 μg/ml Blasticidin S (BS, Kakenseiyaku) and colonies were picked after 10 days.

To detect the presence of HAC as an extrachromosomal element, the BS-resistant cell lines were analyzed by FISH using both α21-I alphoid DNA and BAC vector as probes. Namely, metaphase chromosome spreads were prepared on glass slides after methanol/acetate (3:1) fixation and FISH was carried out according to conventional procedures. For detection of HAC, biotin-labeled alpha 21-I alphoid DNA (11-4) (Ikeno et al. 1994) and digoxigenin-labeled Belo-BAC were used as probes. In dual FISH, biotin-labeled DNA was visualized with FITC conjugated avidin (Vector) and digoxigenin-labeled DNA was visualized with TRITC conjugated anti-digoxigenin (Boehringer Mannheim). Photographs were taken using a CCD camera (Princeton instruments) mounted on a Zeiss microscope. Images were processed using IPLab and Adobe Photoshop 6.0.

One out of 16 transformed cell lines obtained by co-transfection of CMV/α100 and GCH1-BAC (HT/GCH2-10), and three out of 17 cell lines obtained by co-transfection of SV/α50 and GCH1-BAC (one of them is HT/GCH5-18), contained one copy of HAC per nuclei in more than 95% of the inspected cells. In the remaining cell lines, introduced BACs were either integrated into the chromosomes of HT1080 or the signals were undetectable by FISH analyses.

To examine whether the established HACs contained the genomic fragment of the GCH1 gene, four cell lines containing a HAC were further hybridized with probes for GCH1 exon 1 and exons 4-6 (FIG. 3). As a probe for exon 1, 13 kb of biotin-labeled fragment including exon 1 was used, and for probes for exons 4 to 6, 8 kb of digoxigenin-labeled fragments including exons 4, 5 and 6 were used.

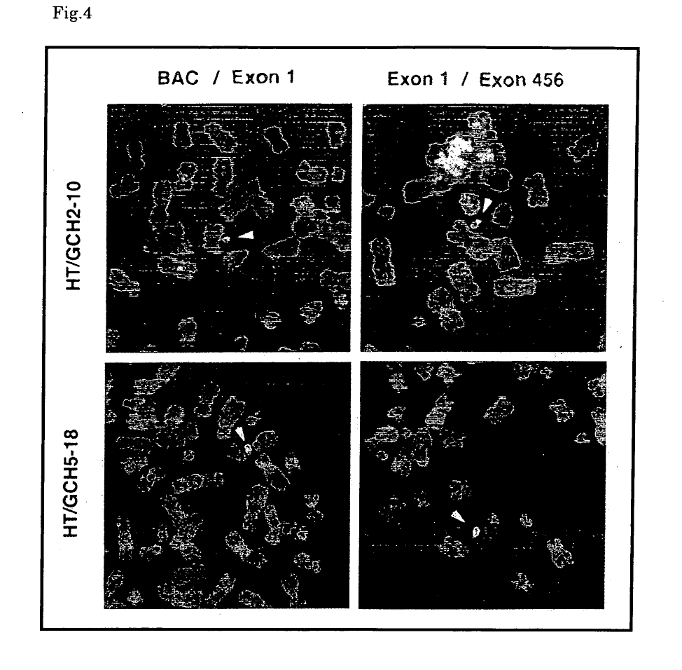

The signals for both probes were detected on a HAC in the HT/GCH2-10 cell line which was generated by co-transfection of CMV/α100 and GCH1-BAC, and on a HAC in the HT/GCH5-18 cell line which was generated by co-transfection of SV/a50 and GCH1-BAC (FIG. 4). The GCH1 signals detected on the HACs were stronger than that of the endogenous gene on the HT1080 chromosomes. As the minority of cells (less than 5%) both in HT/GCH2-10 and HT/GCH5-18 were integrated with the transfected DNA into a chromosome of HT1080, the cell lines were single cell cloned to produce sub-clones containing only one copy of HAC per nuclei and the subsequent re-cloned cell lines were investigated further.

### EXAMPLE 3

### Centromere/Kinetochore Structure and Mitotic Stability of the HACs

To investigate the centromere/kinetochore structure on the HAC, the presence of essential centromere/kinetochore proteins, CENP-A and CENP-E (Palmer et al. 1991; Yen et al. 1991; Howman et al. 2000) was investigated on metaphase chromosomes of HT/GCH2-10 and HT/GCH5-18 by indirect immunofluorescence as follows. Swollen and 1% paraformaldehyde fixed cells were incubated with anti-CENP-A (Ando et al. 2002) or anti-CENP-E (Santa Cruz) antibodies. Antibody localization was visualized with FITC-conjugated anti-mouse IgG. For subsequent FISH analysis, the cells were fixed again with 1% paraformaldehyde and then with methanol/acetate (3:1).

CENP-A and CENP-E signals were detected on HACs in doublets corresponding to the paired sister chromatids, and were similarly detected at the centromeres of all endogenous chromosomes (data not shown).

We examined the mitotic stability of the HACs in the cell line HT/GCH2-10 and HT/GCH5-18 under the non-selective conditions. Maintenance of the HAC in each cell line was measured by FISH analysis on metaphase spreads, which were prepared after 10, 20 and 30 days of culturing. On each sampling day, 50 spreads from each cell line were examined and the percentage of cells that carried the HAC was determined. Namely, the following formula: Nn=N0x(1−R)n, where No is the number of the metaphase spreads containing a HAC under selective conditions, and Nn is the number of metaphase spreads containing a HAC after n days of culture under non-selective conditions. Fish analysis was performed in the same way as the method mentioned above.

After 30 days without selection, 95% of metaphase cells in HT/GCH5-18 and 80% of metaphase cells in HT/GCH2-10 retained the HAC and the number of HAC copies per cell was kept at one under non-selective conditions. The integration into host chromosomes was not observed in either cell line. The rate of chromosome loss per day was calculated from the percentages of cells retaining HAC after 30 days under non-selective conditions. The values were 0.2% and 0.5% for HT/GCH5-18 and HT/GCH2-10, respectively. These results indicated that an active centromere/kinetochore structure was formed on the HACs and that the HACs were stably maintained through mitosis.

### EXAMPLE 4

### DNA Structure of HACs

To determine whether the HACs in HT/GCH2-10 and HT/GCH5-18 were circular or linear, FISH was performed using a telomere sequence and BAC vector as probes. No telomere signal was detected on the HACs when HACs were stained using a BAC vector probe. In contrast, the ends of the chromosomes from the host cell, HT1080, were hybridized as clear speckles. As expected, BAC-derived HACs are likely to be circular in form.

The DNA organization of the HACs was analyzed by restriction digestion of DNA isolated from HT/GCH2-10, HT/GCH5-18 and non-transfected HT1080 cells. The DNA samples (5 μg) were digested with BamHI or StuI for 4 hours followed by conventional gel electrophoresis. The DNA in the gel was transferred to a nylon membrane and hybridized with ³²P labeled DNA probe prepared from GCH1 exon 6 (2.1 kb) and the upstream region of GCH1 (1.4 kb, position 595-1959 in GCH1-BAC).

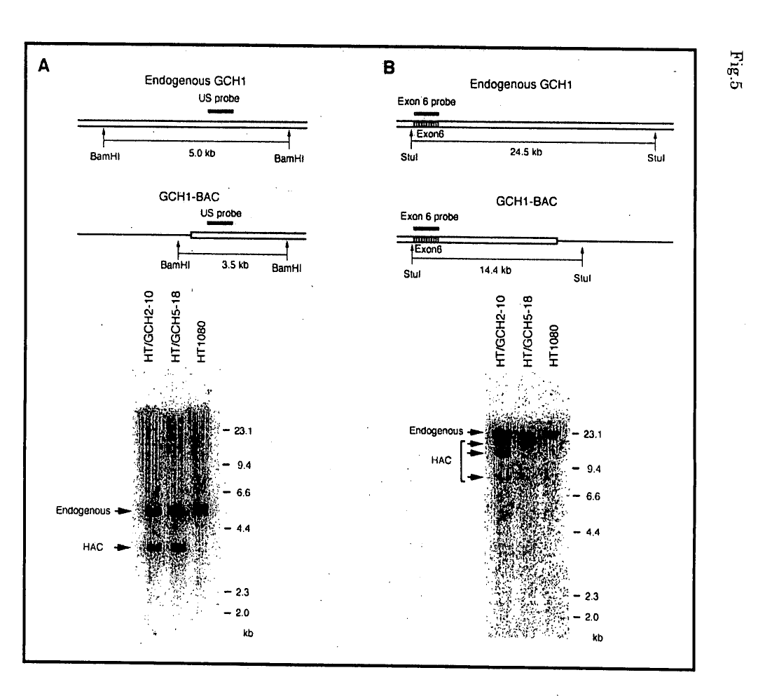

The size of BamHI fragments detected by the US probe were 5.0 kb from the endogenous GCH1 gene and 3.5 kb from GCH1-BAC. The 5.0 and 3.5 kb fragments were detected with DNA from HT/GCH2-10 and HT/GCH5-18 at almost the same signal intensity (FIG. 5(A)). The size of StuI fragments detected by the exon 6 probe were 24.5 kb from the endogenous GCH1 gene and 14.4 kb from GCH1-BAC. The 24.5 and 14.4 kb fragments were detected with DNA from HT/GCH5-18 at almost the same signal intensity, while three fragments heterogeneous in size were detected in addition to the endogenous fragment with the DNA from HT/GCH2-10 (FIG. 5(B)). The results indicated that GCH1-containing HACs in HT/GCH5-18 were established by the assembly of about three copies of transfected GCH1-BAC DNA since the karyo-type of HT1080 cells used in this study is 3n, while HT/GCH2-10 was accompanied by some rearrangements of the terminal region of GCH1 exon 6, but it may also contain three copies of GCH1-BAC as judged by the density of the US band. The internal rearrangements of GCH1 genes were confirmed by RT-PCR analyses of GCH1 transcripts in HT/GCH2-10, which revealed the synthesis of abnormal transcripts (data not shown).

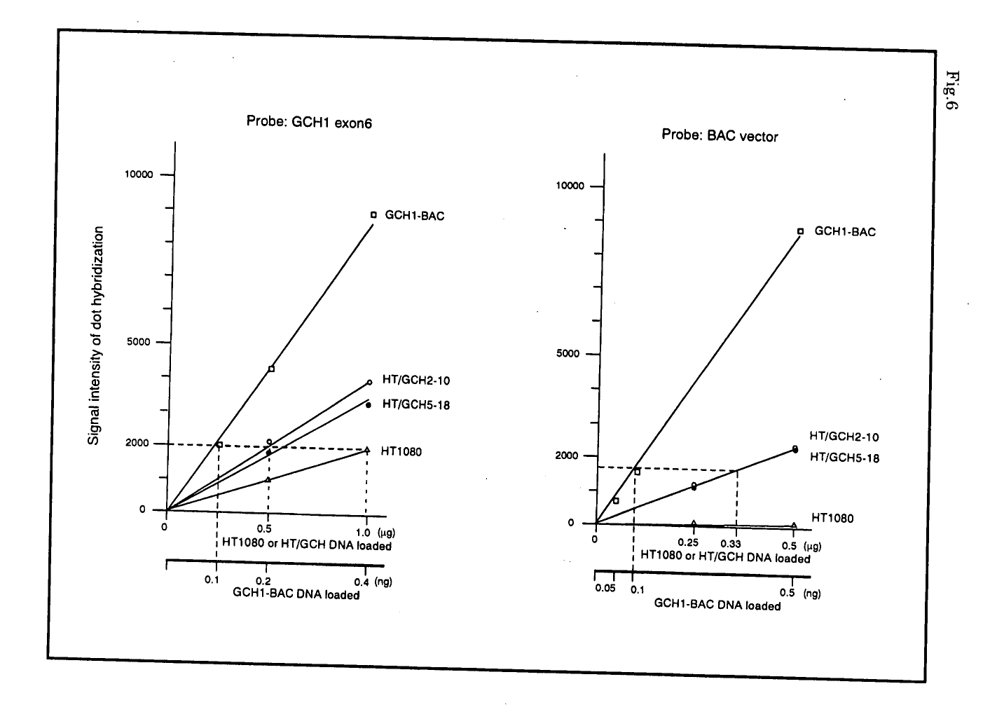

The copy numbers of the GCH1-BAC and the alphoid-BAC in GCH2-10 and GCH5-18 HACs were determined by dot hybridization using GCH1 exon 6 and BAC vector, respectively, as probes. Relative copy numbers of each BAC in the HACs were estimated from the hybridization signal-intensity values, which were determined using each DNA probe and standardized using the values obtained with 0.1 ng GCH1-BAC DNA (FIG. 6). In the case where GCH1 exon 6 was used as the probe, the same hybridization intensity values to that obtained with 0.1 ng GCH1-BAC DNA were obtained with 0.5 μg DNA from both HT/GCH2-10 and HT/GCH5-18, and with 1 μg DNA from HT1080 (FIG. 6, Left). Since HT1080 karyo-type used in this study is 3n, three copies of GCH1 genes occur on its chromosomes, and given that HT/GCH2-10 and HT/GCH5-18 resulted in the same signal values with half the amount DNA as that from HT1080, they must contain six copies of GCH1 genes; three on the chromosomes and three on the HAC. The total copy number of BACs was estimated from the intensity values obtained with the BAC vector probe. The same hybridization intensity values to that obtained with 0.1 ng GCH1-BAC were obtained with 0.33 μg DNA from HT/GCH2-10 and HT/GCH5-18, while HT1080 showed no signal as expected (FIG. 6, Right). Therefore, both HACs have roughly 3-fold more copies of the BAC vector than copies of the GCH1 gene. Thus, copy numbers of the total BAC vectors must be approximately nine per cell; three copies of GCH1 genes are in the form of GCH1-BAC and the remaining six copies of BACs must exist in the form of alphoid-BAC in both HACs.

### EXAMPLE 5

### Transfer of HAC-Containing Cell Lines to Mouse A9 Cells

De novo HAC formation using cloned alphoid DNA has been successful in the human fibrocarcinoma cell line, HT1080. To determine the natural expression of the GCH1 gene in the neural cell line, the HAC that has been established in the HT1080 cell line needs to be transferred into a neural cell line.

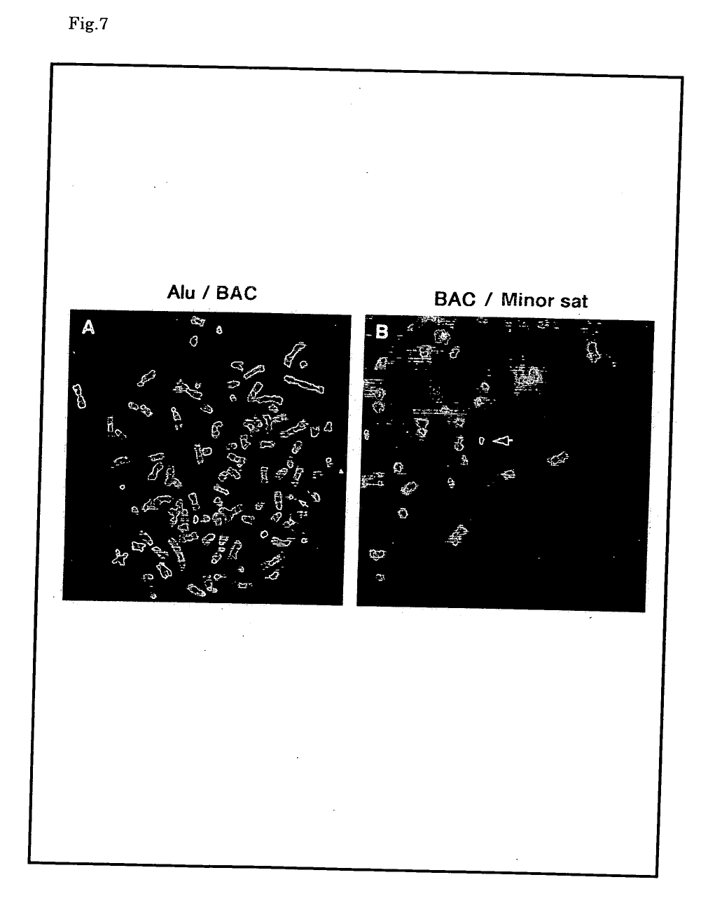

Fusion of the HAC-containing cell lines and mouse A9 cell line was performed using PEG which allows micro-cell mediated chromosome transfer (MMCT) (Fournier et al. 1977). Cell lines containing a HAC (5×105) and mouse A9 cells (5×105) were co-cultivated and fused in PEG/DMSO solution (SIGMA). BS- and Ouabain-resistant cells were selected with 2.5 μg/ml BS and 3 μM Ouabain. BS- and Ouabain-resistant cell lines were analyzed by FISH. Metaphase spreads were hybridized with a BAC vector probe and Alu repeat probe to identify the HACs and human chromosomes, respectively (FIG. 7(A)). One of the fusion cell lines, F/GCH5-18, contained one or two copies of HAC together with eight to ten human chromosomes.

The HACs in the fusion cells were maintained stably during mitotic growth under non-selective conditions with a loss of approximately 1% of the mitotic chromosomes per day (data not shown). The mitotic stability of human chromosomes in mouse cell lines was sometimes caused by the acquisition of minor satellite DNA from the mouse which was localized at the centromere of the mouse chromosomes and may serve as functional centromere sequences (Shen et al. 1997). Therefore the presence of mouse minor satellite DNA on HAC was examined by FISH. Signals of minor satellite DNA were not detected on HAC, while strong signals were detected at the centromeres of mouse chromosomes (FIG. 7(B)). The fusion cell lines were able to form micro-cells under colcemid treatment conditions (data not shown). Therefore, the HACs could be easily transferred to neural cell lines.

### EXAMPLE 6

### GCH1 Expression from HAC

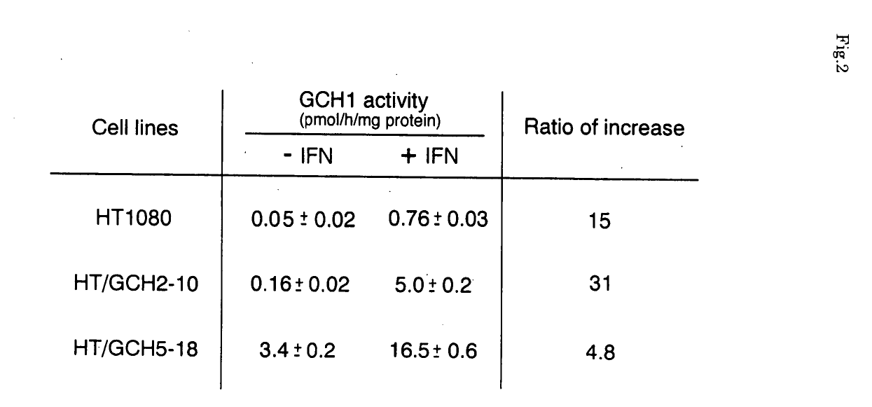

Naturally regulated gene expression was expected from the transgenes in the large genome segments carried by HACs. The GCH1-BAC used in the generation of HACs contained over 100 kb of genomic sequence from the 5′ upstream region of the GCH1 exon 1. Therefore, we have measured GTP cyclohydrolase I (GCH1) activities in HT1080 and the HAC-containing derivatives that were developed from it. It would have been expected from the previous report that the activity of GCH1 would have been hardly detectable in fibroblast cell lines but up-regulated by induction of IFN-γ (Werner et al. 1990). GCH1 activity in HT1080, HT/GCH2-10 and HT/GCH5-18 were analyzed in the presence and absence of IFN-γ induction (FIG. 2). GCH1 activity was measured as follows. Cells were grown in the absence or presence of human IFN-γ at 250 U/ml in culture medium for 48 h. Trypsinized cells were washed in phosphate-buffered saline (PBS), then lysed in 0.1 M Tris-HCl (pH 8.0), 0.3 M KCl, 2.5 mM EDTA, 10% glycerol. GCH1 activity was measured as described (Hibiya et al. 2000).

HT1080 without GCH1-HAC exhibit barely detectable levels of GCH1 activity in the absence of IFN-λ induction, while the activity was increased fifteen times upon the addition of IFN-λ. In HT/GCH2-10 cell line in the absence of IFN-λ induction, the GCH1 level was three times the values of HT1080 without a HAC. After the IFN-λ induction, nearly 30-fold up-regulation was observed. In contrast, GCH1 activity in HT/GCH5-18 was elevated 70-fold in the absence of IFN-λ and addition of IFN-λ further up-regulated the activity 5-fold. In both HT/GCH-HAC cell lines, the GCH1 activities were elevated but differ in degree, possibly reflecting the difference in chromatin structure and/or DNA rearrangements in HACs. They are still susceptible to IFN-λ induction, just like the response of the expression of the GCH1 gene from the authentic chromosome.

As showed above, we obtained HACs containing large DNA fragments with the GCH1 gene (GCH1-HAC) by simple co-transfection methods using alphoid-BAC and GCH1-BAC at a DNA ratio of 1:1. The GCH1-HAC was maintained at one copy after 30 or more rounds of generation under non-selective conditions in spite of being circular in form without telomeres, indicating that HAC replicates once in each cell cycle and is segregated precisely into daughter cells. Therefore, the circular HACs in this study did not cause topological problem, which may result in the abnormal segregation of the circular chromosomes, since the catenated form arose from DNA replication. The HACs were cytologically megabases in size and approximately 10-fold larger than the transfected BAC DNA.

The DNA structure of the HAC was examined to understand the properties and mechanism of de novo generation of HAC. The restriction analysis of the whole area of the GCH1 gene was difficult because almost all rare-cutting enzyme sites in the BAC constructs were subjected to methylation and the cell lines contain the endogenous GCH1 locus. Therefore, we applied restriction analysis to the region corresponding to the junction of the BAC vector and the GCH1 locus. The result showed that GCH1-HACs in the two cell lines contained three copies of the GCH1-BAC and six copies of the alphoid-BAC as components (FIG. 5, 6). Although the exact mechanism of the formation of the HAC remains unknown, GCH1-HAC was composed of multimer of the input DNA, which was similar to the HAC generated by alphoid-YAC (Ikeno et al. 1998) or alphoid-BAC alone (data not shown) as an input element. The fact that the formation of HAC was accompanied by assembly of the distinct BACs indicated that the multimerization of BAC molecules might be mediated by non-specific recombination of input BAC in addition to the amplification of BAC DNA itself.

The generation of the HAC containing large human genomic DNA was previously reported using a 140 kb or 162 kb HPRT locus (Grimas et al. 2001; Mejia et al. 2001). They obtained the HAC containing HPRT gene in the HPRT-deficient HT1080 cell lines in HAT medium depending on complementation. The feasibility of such an approach for genes with tissue and stage specific expression (i.e. not house-keeping gene) will be low in HT1080 cells. In this study, we found that as short as 50 kb of a21-I alphoid DNA in BAC was able to generate HACs (centromere/kinetochore) and that the BACs containing large transgene without a selection markers could be incorporated efficiently into the HAC, since 50% of the HACs included the transgene. Thus, HACs containing any large genomic region of interest could be generated using alphoid-BAC containing 50 kb of alphoid DNA and a readily available BAC library without any modification. Intactness of the incorporated transgenes may be checked after HAC generation.

The selection of transformants with CMV promoter-driven Bsd gene increase the number of BS-resistant cells, but the FISH signals for HACs or integrated loci on the host chromosomes were not found in the majority of transformants (FIG. 1). Southern hybridization analyses using alphoid and BAC vector sequences as probes indicated that these cell lines have Bsd genes only integrated in the chromosomes. Thus, the selection marker, driven by a high expression promoter, was not suitable for the screening of HAC-containing cell lines.

Gene expression was affected by chromosome structure. The insertion of a transgene into a chromosome often results in stably inherited gene silencing in a clonal sub-population of the cells, a phenomenon commonly known as position effect variegation (PEV) (Karpen 1994). Recent molecular analysis showed that methylation of histone H3 on lysine 9 contributes to the targeting of HP1 to the chromatin and results in heterochromatinization and the silencing of gene expression (Platero et al. 1995; Bannister et al. 2001; Lachner et al. 2001). Gene silencing at or near the centromere/kinetochore in yeast and fly was also reported (Karpen & Allshire 1997) and was expected to occur in mammalian cells. We have recently demonstrated that in the alphoid array of a HAC, once centomere/kinetochore structure was formed, the expression of the short marker genes inserted into the HACs were repressed strongly even if they were driven by strong promoters (Abe et al. submitted). Thus, to get expression of transgenes in HACs, we will need to solve the topological problem of the genes in relation to the centromere/kinetochore structure.

The GCH1 gene expression from HAC might be correlated with the chromatin structure at or near the GCH1 locus. The present inventors addressed whether the centromere/kinetochore structure was formed on only the alphoid array or whether it spread into the GCH1 locus. Since CENP-A is an essential protein for a functional centromere/kinetochore and constitutes the histone component for centromere specific nucleosomes (Palmer et al. 1991; Howman et al. 2000), we analyzed the chromatin structure on HAC by ChIP using anti-CENP-A antibody (Ando et al. 2002). ChIP method was performed as follows. The nuclei of HT/GCH5-18 cells (5×107) were isolated and dissolved in WB (20 mM HEPES (pH 8.0), 20 mM KCl, 0.5 mM EDTA, 0.5 mM dithiothreitol, 0.05 mM phenylmethylsulfonyl fluoride). After digestion with MNase, solubilized chromatin was immunoprecipitated using anti-CENP-A antibody as described previously (Ando et al. 2002).

In such analyses using HeLa and HT1080 cells, the alphoid array was enriched to 60-80% in total immunoprecipitated DNA. The alphoid array in the GCH1-HAC was also enriched by anti-CENP-A antibody, while the GCH1 region was not. In contrast, the BAC vector sequence about 3 kb away from the alphoid sequence was also immunoprecipitated, indicating that the centromere/kinetochore structure was formed on the alphoid array and spread to flanking non-alphoid region (data not shown). The invasion of the GCH1 locus in the HAC by centromere/kinetochore structure, was prevented by an as yet unknown protection mechanism that probably resides in the upstream regulatory sequence.

GCH1 encodes the first and rate-limiting enzyme for the biosynthetic pathway of tetrahydrobiopterin (Nichol et al 1985), the co-factor of aromatic amino acid hydroxylase (PAH, TH, TPH) as well as nitric oxide synthase (NOS) and is present in higher organisms (Kaufman 1993). The GCH1 gene is a causative gene for dopamine deficiency in dopa responsive dystonia (DRD/Segawa's disease) (Ichinose et al. 1994). Deficiency of GCH1 in conjunction with a mutation in the TH gene results in severe early-onset dystonia/parkinsonism (Ichinose et al. 1999).

Although only limited analyses have been performed on the upstream regulatory sequence of the human and mouse GCH1 gene, it has been reported that the CCAAT and TATA boxes are conserved (Ichinose et al. 1995; Hubbard et al. 2002). It was established that GCH1 gene expression could be induced by IFN-γ in various rodent and human cells (Werner et al. 1990). However, the exact mechanism involved in IFN-γ signal transduction is yet unknown.

Some gene expression, such as that for human beta-globin, was regulated by locus control regions (LCRs) responsible for initiating and maintaining a stable tissue-specific open chromatin structure (Festentein et al. 1996; Milot et al. 1996). The GCH1-HACs used in this study carry a 180 kb genomic fragment containing the GCH1 gene, and therefore may contain the regulatory sequences required for tissue specific expression and for prevention of the silencing effect of the flanking centromere. The expression of the GCH1 gene from HAC was measured by GTP cyclohydrolase I activity in the presence and absence of IFN-γ (FIG. 2). Activity in the HAC-containing cell line, HT/GCH2-10, was slightly higher than the activity obtained with HT1080 in the absence of IFN-γ. Addition of IFN-γ increased the GCH1 activity approximately 30-fold. In another cell line, HT/GCH5-18, which also carries twice the number of GCH1 genes as HT1080, showed 70-fold higher enzyme activity than TH1080 in the absence of IFN-γ and the activity was further increased 5-fold by IFN-γ induction. The small difference in values between HT/GCH2-10 and HT1080 may correspond to the small copy numbers of intact GCH1 genes, since it seems that some copies of GCH1 genes on GCH2-10 HAC have the structural abnormality described in the Results section. These results indicated that although the gene expression of GCH1 may be affected by the difference of chromatin structure assembled on the GCH1 locus in the HAC, the genes still responded to IFN-γ. The final levels of GCH1 activity after IFN-γ induction in the cell lines were still repressed and kept to a similar order of magnitude. This might indicate the presence of complex cellular regulation systems to maintain the GCH1 activity in the proper range. The GCH1-HAC should prove to be a suitable system to understand the complex regulatory mechanisms of GCH1 expression in vivo.

The adeno-associated virus (AAV) vector was often used for gene therapy in the helper virus-dependent manner for productive infection. The AAV vector has limited cloning capacity that usually carry cDNA without original regulatory sequence for gene expression (Dong et al. 1996). GCH1 is necessary for efficient dopamine production together with tyrosine hydroxylase (TH) and aromatic-L-amino-acid decarboxylase (AADC). Expression of these three enzymes from the AAV vector in the striatum resulted in relatively long-term behavioral recovery in a primate model of Parkinson's disease (Muramatsu et al. 2002).

Recently, an Epstein-Barr virus (EBV)-based episomal vector was reported that was capable of transferring a HPRT gene (115 kb) to some mammalian cell lines, in which the expression was not silenced (Wade-Martins et al. 2000). However, EBV-based vectors are lost more rapidly than HAC in the absence of selection and their replication is reliant on the presence of the viral trans-activator, EBNA1. Safety in the clinical gene therapy with EBV vectors requires further investigation. HACs may overcome the above problems as gene transfer vectors and have further advantages in term of safety. HACs carried a long genomic locus in this study were maintained extrachromosomally, and expressed regulated level of genes for long periods. Therefore, the HAC containing TH, AADC and GCH1 may offer a potential therapeutic strategy for Parkinson's disease.

However, the low efficiency of de novo generation of HACs from BACs, requirement of the limited cell line for generation of HACs and the large size of HACs presents a difficulty in the delivery of HACs to cells or tissues at required sites. To utilize the HAC as a gene transfer vector, the HAC that has been established in HT1080 needs to be transferred into suitable cell lines. The HAC could be transferred by MMCT using the mouse A9 cells, which enable the formation of micro-cells (Fournier et al. 1977). The present inventors have established mouse A9 cell lines, which maintained HACs stably in mitotic growth under non-selective conditions without detectable structural changes in the HAC. The HACs would be easily transferrable from A9 to other cell lines.

We have demonstrated in this study the generation of a HAC containing the GCH1 gene, together with its original regulatory region, from a GCH1-BAC by co-transfection with the alphoid-BAC. The GCH1-HACs expressed GCH1 genes in regulated manner and thus proved to be a good system to study regulatory mechanism of GCH1 gene in vivo. Further study on the GCH1-HACs will reveal the minimum number of alphoid arrays used to assemble centromere/kinetochore, the structure of the upstream region required for regulated expression of the GCH1 gene and sites of action of transcription factors on the regulatory region. Results we have obtained also indicated that the GCH1-HAC may also serve as a gene delivery tool in animal models or therapeutic trials in the future.

### EXAMPLE 7

### Transfer of HACs into ES Cells by Micro-Cell Mediated Chromosome Transfer

HT1080 cells containing HACs retaining GCH1 gene were transferred to mouse A9 cells by a cell fusion method. First of all, by the same procedure as in Example 5, HAC-containing cell lines (HT/GCH2-10) were fused to mouse A9 cells, and BS- and Ouabain-resistant cell lines were selected. To the selected sell lines F(A9/2-10)₄, colcemid was added so that the final concentration became 0.05 μg/ml, followed by culturing at 37° C. under conditions of 5% CO₂ for 72 hours. Cells were collected by trypsinization and suspended in a D-MEM medium without serum. Cytochalasin B was added so that the concentration became 20 μg/ml and left at 37° C. for 5 minutes, followed by adding an equal amount of Percol which had been kept at 37° C. in advance. Then, micro-nuclei were collected by centrifugation (15,000 rpm for 90 mins). The collected micro-nuclei were suspended in a D-MEM medium without serum, followed by centrifugation (2,000 rpm for 5 mins) again. The obtained precipitates (micro-nuclei) were suspended in a D-MEM medium without serum again. After repeating this operation twice, to the precipitates including micro-nuclei, ES cells TT2 (C57BL/6×CBA), which were collected by trypsinization, were added, followed by centrifugation (1,500 rpm for 5 minutes). Thus, cells and micro-nuclei were precipitated. After removing supernatant, 1 ml D-MEM medium without serum was added so as to suspend the precipitates, which was kept in this state for 10 minutes (at room temperature). Then, cells and micro-nuclei were precipitated by centrifugation (1,500 rpm for 5 minutes) and the supernatant was removed, followed by adding 1 ml of PEG1500 (Roche) so as to suspend the precipitates. After leaving it for 90 seconds at room temperature, 5 ml D-MEM medium without serum was added and cells were collected by centrifugation (1,000 rpm and 5 min).

To the collected cells, 10 ml of D-MEM medium without serum was added, followed by washing by centrifugation at 1,000 rpm for 5 minutes twice. Precipitates after washing were suspended in an ESM medium (D-MEM+non-essential amino acid (Invitrogen)+0.1 mM β-mercaptoethanol+10³ U/ml ESGRO (Chemicon)+nucleoside). The obtained cell suspension was plated on feeder cell SLB (provided by Dr. Yuzo Kadokawa at Fujita Health University School of Medicine) which were treated with mitomycin C so as to stop the proliferation. After 24 hours the culturing was started, a medium was replaced with an ESM medium containing blastcidin S so that the final concentration was 4 μg/ml and the culturing was continued. After five days the selection operation was started, the medium was replaced with an ESM medium (4 μg/ml blastcidin S, 1×HAT (Sigma)), and then the culturing was continued further for five days.

The resultant colony was isolated and plated on feeder cells SLB (24 well culture dish) which were treated with mitomycin C to stop the proliferation. From proliferated cells, cell lines containing BAC DNA were selected by PCR, and subjected to FISH analysis using an alphoid DNA, a BAC vector, a GCH1 gene and a mouse minor satellite DNA as probes (see Examples 2 and 5).

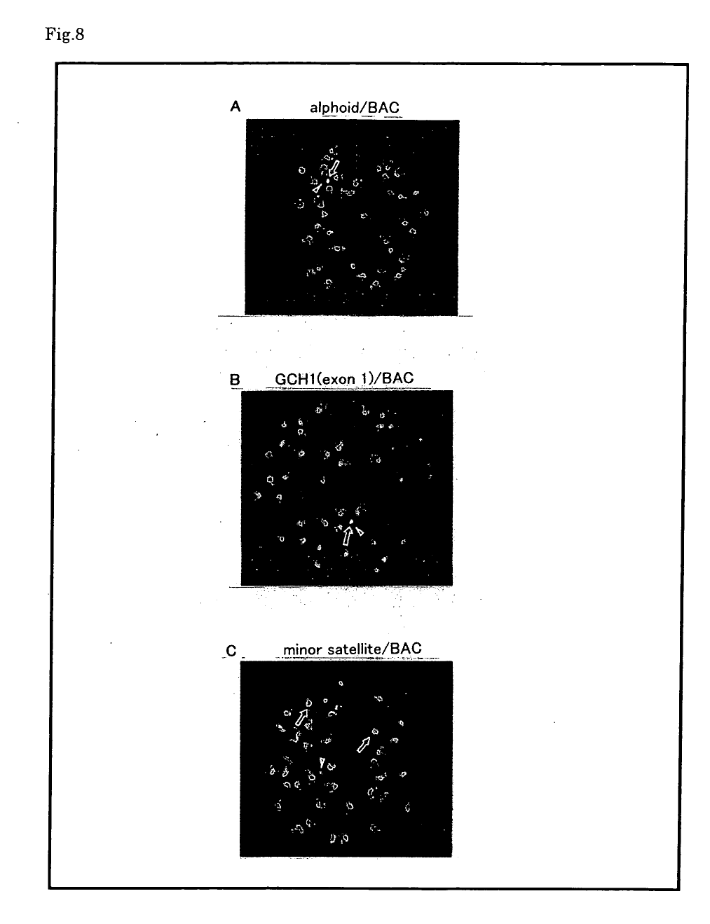

FIG. 8A shows the result of the FISH analysis using an alphoid DNA and a BAC vector as probes. Green indicates a signal of the alphoid DNA (arrow) and red indicates a signal of the BAC vector (arrow head). It is shown that the isolated ES cells contain one copy of HAC and maintain a normal nucleus type.

FIG. 8B shows the result of FISH analysis using an exon 1 region of a human GCH1 gene and a BAC vector as probes. A green signal (arrow) of GCH1 gene and a red signal (arrow head) of the BAC vector were simultaneously detected on the HAC.

FIG. 8C shows the result of FISH analysis using a mouse minor satellite DNA and a BAC vector as probes. On the HAC, a signal (a part of which is shown by an arrow) of the mouse minor satellite DNA were not detected. Note here that an arrow head show a signal of the BAC vector.

### EXAMPLE 8

### Stability of HAC in ES Cells

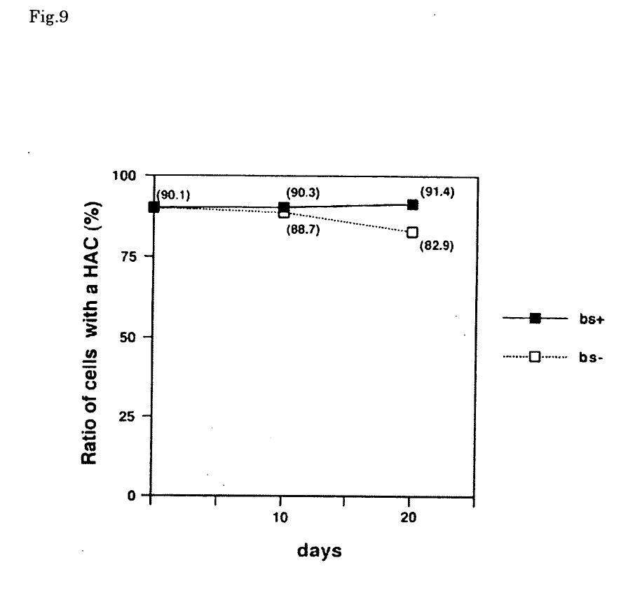

The stability of HAC in ES cells was analyzed by culturing in the absence of selective agents for a long time. The HAC-containing ES cells obtained in Example 7 were cultured (20 days) both in the presence and absence of blastcidin S, followed by calculating the rate of HAC-containing cells by FISH analysis. FIG. 9 shows the result of the analysis. Even after a long term culturing in the absence of agent, 80% or more of cells retain HAC in a state of one copy. When the loss rate of chromosomes per cell division was calculated, it was 0.2%, which showed substantially the same level of stability as in the case of HAC in HT/GCH2-10 cells. Note here that the loss rate R of chromosomes was calculated from the following equation:
Nₙ=N₀×(1−R)ⁿ

### EXAMPLE 9

### Construction of Human Artificial Chromosome (HAC) Using Yeast Artificial Chromosome (YAC)

Human artificial chromosome containing an entire region of β-globin gene group (cluster) of the human chromosome 11 by using YAC as a precursor was constructed by the following procedure. The precursors used follow.

(9-1) Precursor YAC

A201F4.3: 150 kb of YAC containing human β globin gene locus in which the right arm portion of A201F4 was modified and PGKneo was inserted (provided from Keiji Tanimono, Douglas Engel, Nucleic Acid Research, 27; 3130-3137).

7c5hTEL: an artificial chromosome precursor YAC including about 80 kb of alpha-satellite array (α21-I) derived from the human chromosome 21 alphoid region and a marker gene SVbsr, and having yeast telomere sequences at both ends and human telomere sequence inside thereof. Yeast containing 7c5hTEL (Saccaromyces serevisiae EPY 305-5b α7C5hTEL) was disposed with Agency of Industrial Science and Tehnology, National Institute of Bioscience and Human Technology in Ministry of International Trade and Industry (at present, National Institute of Advanced Industrial Science and Technology, International Patent Organism Depositary, of which address is Chuo No. 6, 1-3, Higashi 1-chome, Tsukuba-shi, Ibaraki-ken, 305-8566, Japan) on Aug. 14, 1996 (deposition No: FERM BP-5625), and 7c5Htel is prepared from this yeast cell line. As to the production method of the yeast cell line, see, for example, Published Japanese translation of a PCT application No. 2000-517182.

F61: a tetracycline-induced expression system cell established by introducing pTet-OFF (CLONTECH) into HT1080 and by the selection of G418.

(9-2) Purification of Yeast Artificial Chromosome

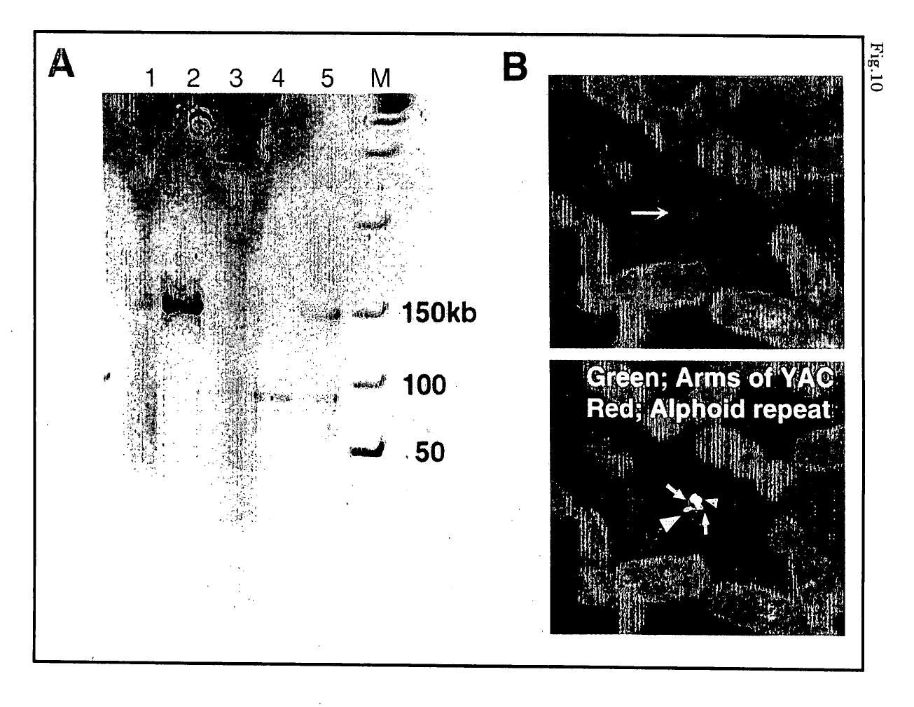

Pulsed Field Gel Electrophoresis (PFGE) was carried out by the following procedure so as to isolate two kinds of yeast artificial chromosomes (A201F4.3 and 7c5hTEL), respectively. PFGE was carried out on 0.7% agarose gel under the conditions of 0.5×TBE, 180 Volt and 15 second pulse for 15 hours by using Gene Navigator (Amersham Pharmacia Biotech). YAC DNA isolated from the PFGE gel was transferred to agarose gel with 1% low melting point by electrophoresis, and then this gel was immersed in a buffer solution of 10 mM Tris (pH 8.0), 1 mM EDTA and 100 mM NaCl for 16 hours. 100 μg E. coli tRNA was added to YAC DNA (0.3 μg/0.3 ml), which was heated at 70° C. for 10 minutes so as to melt the gel. 30 U β agarase (Sigma) was added and reacted at 42° C. for 2 hours to digest the agarose. These were subjected to PFGE so as to confirm bands of 7c5hTEL (90 kb) and A201F4.3 (150 kb) (see FIG. 10A).

(9-3) Introduction of YAC

0.3 μg each of the purified 7c5hTEL and A201F4.3 were mixed and 60 μl of Superfect (Qiagen) was added, followed by gently mixing thereof so as to cause a reaction at room temperature for 10 minutes. The reacted solution was added to F61 cells. 90 minutes later, the culture solution (10% FBS (Trace Scientific Ltd., Noble Park, Australia) in D-MEM: Dulbecco's Modified Eagle Medium (Invitrogen Corp., Carlsbad, Calif., USA)) was replaced with a new one. 72 hours later, resistance cell lines were selectively cultured on 8 μg/ml Blasticidin S added medium so as to isolate the transformed cell lines. As a result, 19 transformed cell lines were obtained.

### EXAMPLE 10

### Cytogenetic Analysis of Transformed Cell Line

The obtained transformed cell lines were subjected to FISH analysis by using an α21-I probe (alphoid probe obtained by labeling a DNA fragment of SEQ ID NO: 3 with digoxigenin) and a probe of the arm portion of YAC (obtained by labeling about 8 kb of DNA fragment (SEQ ID NO: 4) obtained by XhoI-cutting a pYAC5 vector (Dr. Maynard V. Olson (Washington University)) with biotin). As a result, it was observed that mini chromosome was formed in one transformed cell line and signals was included in both α21-I and the arm portion of YAC in this mini chromosome (see FIG. 10B). In the rest of clones, signals were observed on the host chromosome or no signals were detected.

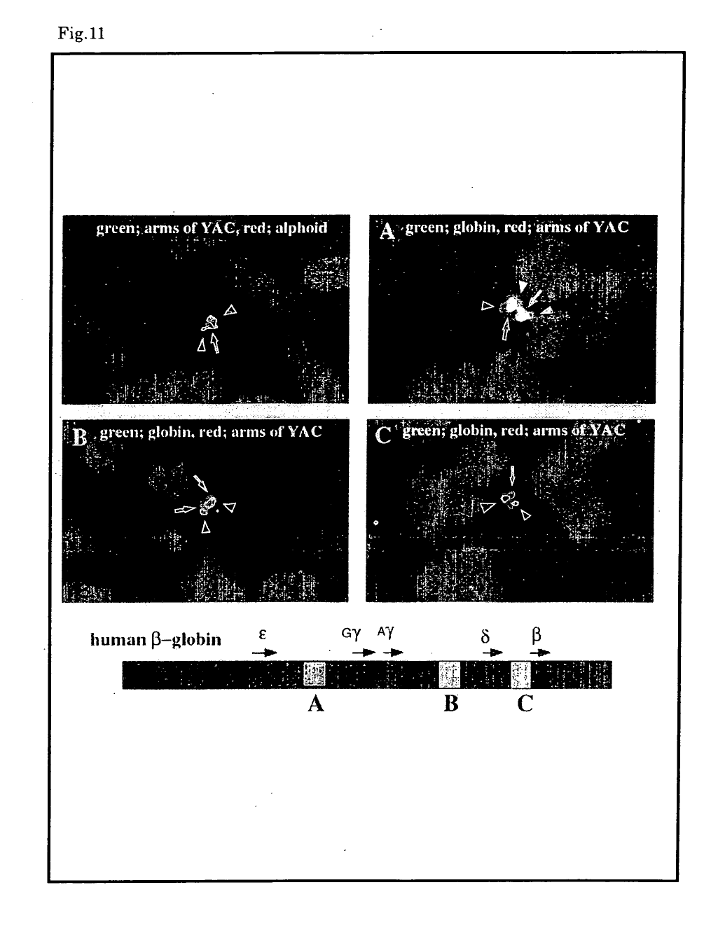

On the other hand, when the transformed cell lines including mini chromosome were subjected to FISH analysis by using three kinds of probes (SEQ ID NO: 5, SEQ ID NO: 6 and SEQ ID NO: 7) each recognizing different sites of a human β globin cluster non-coding region, signal from each probe was observed on the mini chromosome (see FIG. 11). Note here that each probe was obtained by subjecting it to PCR (25 cycles each cycle including 96° C. for 30 seconds, 58° C. for 40 seconds and 72° C. for 10 minutes) by using A201F4.3 as a template and by using the following primers and labeling the resultant amplified DNA with biotin.

Primer for probe shown in SEQ ID NO: 5

| | |
|---|---|
| Sense: | |
| aagaccagatagtacagggcctggctac | (SEQ ID NO:10) |
| Antisense: | |
| aagattattcaaggttactatgaacacc | (SEQ ID NO:11) |

Primer for probe shown in SEQ ID NO: 6

| | |
|---|---|
| Sense: | |
| tgctaatgcttcatctagaaacttatatcctttaa | (SEQ ID NO:12) |
| ttc | |
| Antisense: | |
| tttccactcgagccaaccaggaattcggcagttac | (SEQ ID NO:13) |

Primer for probe shown in SEQ ID NO: 7

| | |
|---|---|
| Sense: | |
| gtgtaagaaggttctctagaggctctacagatagg | (SEQ ID NO:14) |
| gag | |
| Antisense: | |
| aagcagcacttgactcgagtatttttatacatgct | (SEQ ID NO:15) |
| ctac | |

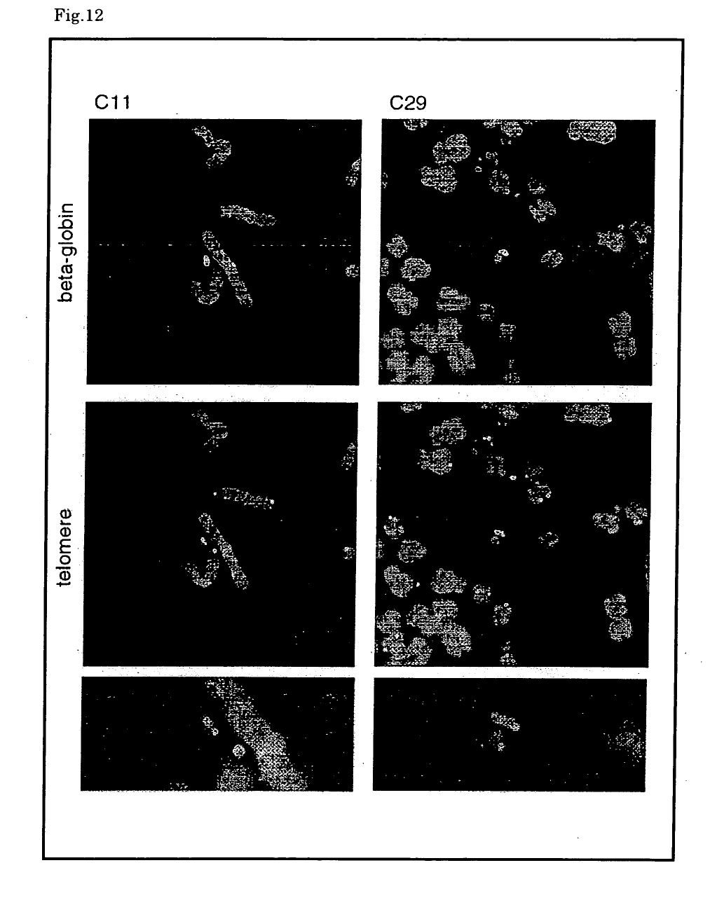

Furthermore, in the FISH analysis using a digoxigenin-labeled telomere repeat sequence (about 500 bp of sequence consisting of repeat sequences of SEQ ID NO: 8) as a probe, two points or four points of signals of telomere were observed on the mini chromosome (see FIG. 12).

From the results mentioned above, YAC including an alpha-satellite array and YAC including human β globin cluster entire region were introduced into HT1080 cells, whereby it was confirmed that mini chromosome (human artificial chromosome) retaining an entire region of human β-globin cluster could be constructed.

### EXAMPLE 11

### Analysis of Macro Structure of Mini Chromosome Using Fiber FISH

Mouse A9 cells and cells with mini chromosomes (1×10⁶ each) were plated on a culture dish and 3 ml of 50% PEG (SIGMA) was added thereto and cultured for one minute. Then, they were cultured in a selection medium containing 10 μM Oubain and 5 μg/ml of Blasticidin S so as to obtain resistant transformed cells to Oubain and Blasticidin S. When FISH analysis was carried out as mentioned above, it was confirmed that there were transformed cell lines in which mini chromosomes were contained and the remaining chromosomes were derived from the mouse. These transformed cell lines were subjected to FISH analysis using an alphoid probe (SEQ ID NO: 3) and a β globin probe (mixture of SEQ ID NO: 5, SEQ ID NO: 6 and SEQ ID NO: 9). Note here that the probe of SEQ ID NO: 9 was a biotin-labeled DNA fragment which was amplified by PCR (25 cycles each cycle including 96° C. for 30 seconds, 58° C. for 40 seconds and 72° C. for 10 minutes) using A201F4.3 as a template and the following primers.

Sense: gtatacatacatacctgaatatg (SEQ ID NO: 16)

Antisense: tgtaggctgaagacgttaaaagaaacac (SEQ ID NO: 17)

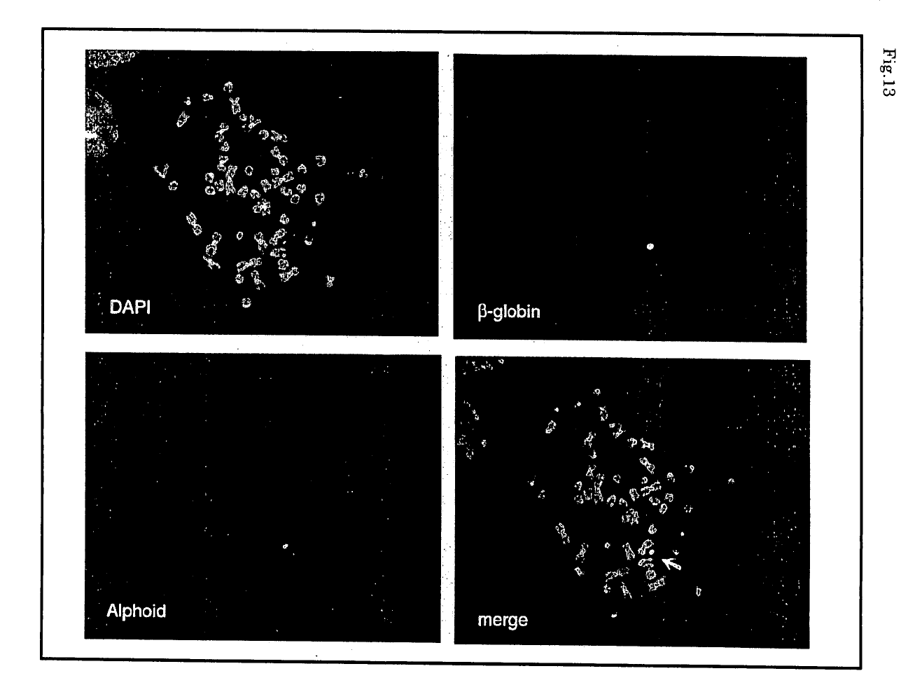

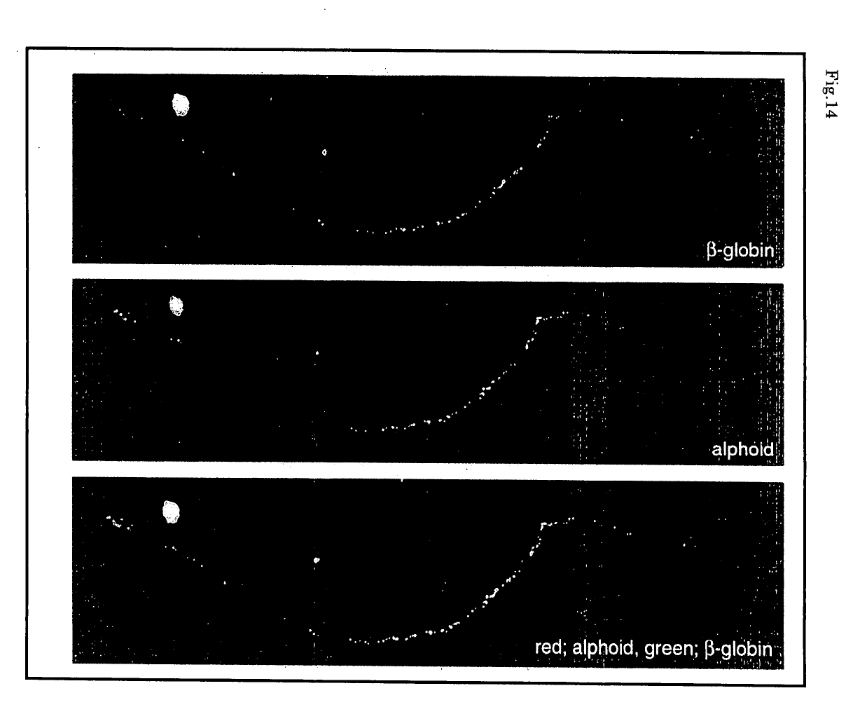

As a result of the FISH analysis, since signals of the alpha-satellite array are not observed on the chromosome other than the mini chromosome (see FIG. 13), fiber FISH analysis of alpha-satellite array of the mini chromosome was possible. When the fiber FISH analysis was carried out, it was confirmed that a plurality of signals of globin and alphoid array were arranged irregularly in the mini chromosome (FIG. 14).

### EXAMPLE 12

### Analysis of Transcription Amount of Target Gene from HAC

Then, the transcription amount of globin genes in HAC-containing cells retaining β-globin gene was analyzed.

By the same procedures as in Example 9, 7c5hTEL and A201F4.3 were introduced into leukocyte K562 cells (ATCC CCL-243) so as to obtain HAC-containing cells retaining β-globin gene (HAC-containing K562 cells). The expression states of HAC-containing K562 cells and globin gene in HAC-containing HT1080 cells were analyzed by using the transcription amount of Gγ globin as an index as follows. Note here that HT1080 cells and K562 cells before the introduction operation of 7c5hTEL and A201F4.3, that is, HT1080 cells and K562 cells which do not contain HACs, were used as a control for comparison.

First of all, RNA was extracted by a conventional method from each cell, and cDNA was synthesized by using reverse transcriptase of MMLV and an Oligo (dT) 15 primer. The thus obtained cDNA was, as a template, subjected to RT-PCR using the following primer set (exon 2 and exon 3 of Gγ globin).

| | |
|---|---|
| Sense primer: | |
| gatgccataaagcacctggatg | (SEQ ID NO:18) |
| Antisense primer: | |
| ttgcagaataaagcctatccttga | (SEQ ID NO:19) |

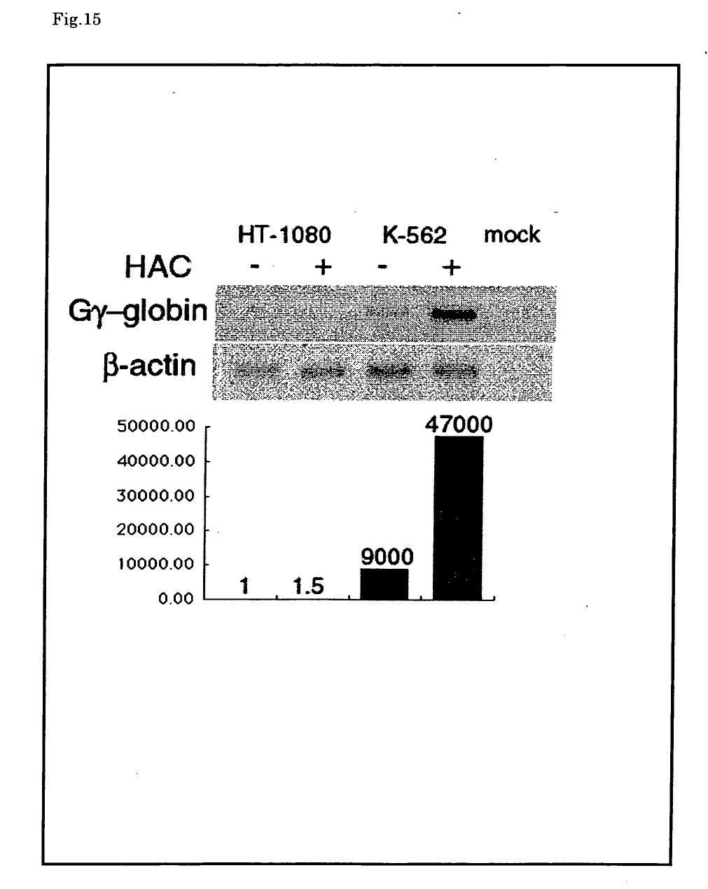

The results of RT-PCR were shown in the upper part of FIG. 15. Note here that the results of RT-PCR which were similarly carried out by using the following primers specific to β-actin gene are also shown.

| | |
|---|---|
| Sense primer: | |
| tcacccacactgtgcccatctacga | (SEQ ID NO:20) |
| Antisense primer: | |
| cagcggaaccgctcattgccaatgg | (SEQ ID NO:21) |

On the other hand, the transcription amount of each sample of Gγ globin genes was quantified by a real-time PCR. The real-time PCR was carried out by using ABI PRISM 7700 (ABI, Applied Bio systems Inc.) and Qiagen QuantiTect SYBR Green PCR kit (Cat 204143). Furthermore, as the primer used for amplification reaction, the above-mentioned primers were used. Note here that the transcription amount of β actin gene in each sample was calculated and the difference of the numbers of cells between the samples was corrected based on the calculated transcription amount.

The lower part of FIG. 15 shows the analysis results by the real-time PCR. Note here that the transcription amount of Gγ globin in each sample was expressed as a relative value when the trnscription amount of HT1080 without containing HAC was 1. By the introduction of HAC, the amount of expression of Gγ globin became 1.5 times when the target cell was HT1080. Meanshile, when the target cell was K-562, the expression amount became 5 time or more. Thus, regardless of target cell to be used, the expression of Gγ globin from the introduced HAC, that is, the expression of foreign gene contained in HAC was confirmed. In particular, it was shown that in a case where K-562 was used, foreign genes could be expressed with extremely high activity.

### EXAMPLE 13

### Creation of HAC-Containing Mouse (Chimeric Mouse)

Cell lines established by culturing ES cells containing HAC (HAC-containing ES cell lines TT2/GCH2-10) obtained in Example 7 were transfused into 8 cell-stage embryo or blastocyst stage embryo collected from ICR mouse (CLEA Japan Inc.) by an injection method, and ES cell-introduced embryo was transplanted into a provisional parent. Thereafter, a child mouse was born by natural childbirth. From the mouse 24 hours after its birth, organs (brain, heart, thymus, liver, spleen and kidney) were isolated and genomic DNAs were prepared with respect to each organ. The obtained DNA was subjected to PCR by using FastStart Taq DNA polymerase (Roche) so as to detect DNA derived from BAC. The sequence of primer to be used and cycle (reaction conditions) are as follows.

| | |
|---|---|
| BAC3a primer: | |
| catcgtctctctgaaaaatcg | (SEQ ID NO:22) |
| CHIPBAC3b primer: | |
| aggaaacagcaaaactgtgac | (SEQ ID NO:23) |

Cycle: 95° C., 4 minutes×1; 95° C., 15 seconds; 55° C., 10 seconds; 72° C., 30 seconds×35; and 72° C., 9 minutes×1

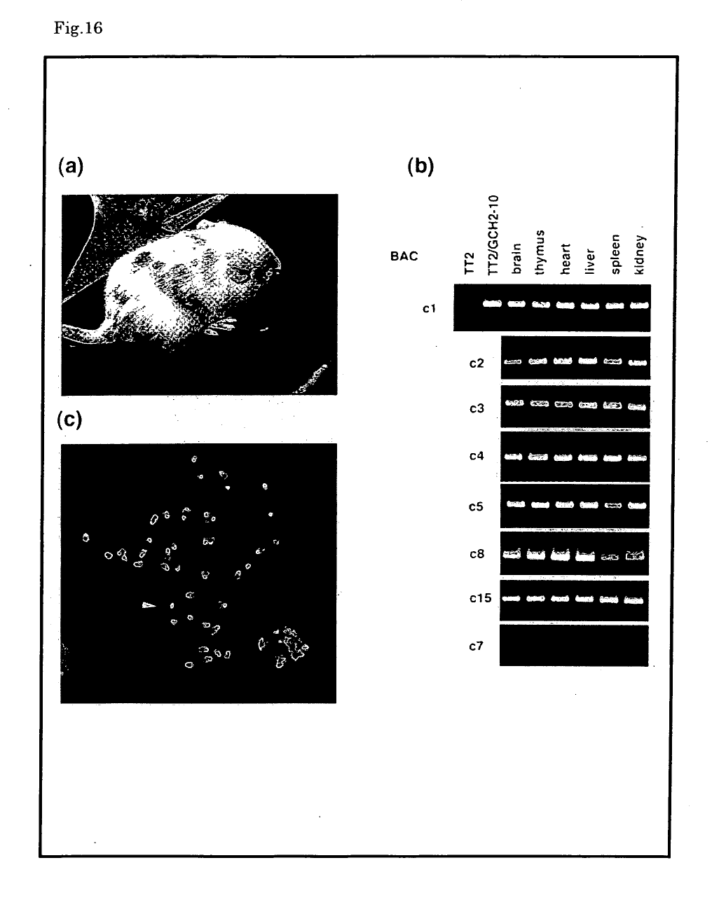

The results of analysis by PCR are shown in FIG. 16 (b). As a result of the analysis of 15 mice, as shown in this figure, in 7 mice, BAC DNA were detected in all organs.

Then, to confirm the presence of GCH-HAC in a chimeric mouse individual body created by using HAC-containing ES cells, chromosome sample of cell division stage was made and subjected to FISH analysis. First of all, a chimeric mouse excluding head portion and visceral organs was washed with PBS and stripped, and then kept at 37° C. for 1 hour in the presence of 0.05% trypsin/1 mM EDTA. Cells trypsinized from the strip were collected by centrifugation and washed with DMEM medium including 10% FCS twice. The cells were floated in DMEM containing 10% FCS again and cultured in the presence of 5% CO² at 37° C. To the culture, which was increased approximately to confluent, TN16 was added and synchronized to the division stage, followed by making chromosome sample of cell division stage.

As a result of FISH analysis using the alphoid array and the BAC vector sequence as probes, artificial chromosome (GCH-HAC) was confirmed in cells derived from chimeric mouse (derived from ES cells) (see FIG. 16 (c)). Note here that FIG. 16 (a) shows the obtained chimeric mouse. It could be confirmed that it was a chimeric mouse from a hair color.

### EXAMPLE 14

### Transfer of HAC to XO Nuclear Type ES Cell Lines and Creation of Chimeric Mouse

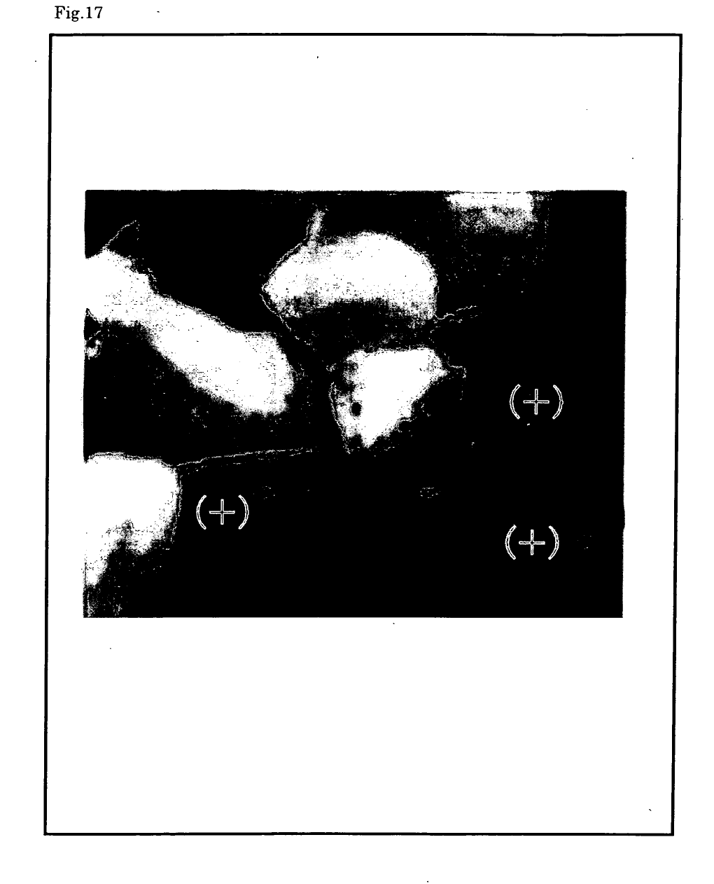

By the same procedures as shown in Example 7, HAC was transferred into the mouse ES cells by MMCT. In Example 7, XY nuclear type ES cells were used in Example 7, but in this Example, XO nuclear type ES cells TT2-F (provided by Dr. Aizawa) was used. When cells obtained after MMCT treatment were subjected to FISH analysis, some cells contained HACs as expected (data are not shown). The thus obtained HAC-containing ES cells were cultured so as to establish cell lines. Thereafter, by using these cell lines, a chimeric mouse was attempted to produce by the same procedure as in Example 13. As a result, as shown in FIG. 17, a chimeric mouse (female) with mosaic hair color was obtained.

### EXAMPLE 15

### Construction of Mammalian Artificial Chromosome Having Gene Insertion Site

Artificial chromosome including a gene insertion site and human β globin LCR as a candidate of an insulator sequence was constructed, and the effect of the insulator sequence in the artificial chromosome was verified.

15-1. DNA Construct

(1) Human β Globin LCR

20836 kb (GenBank data base NG000007: 4818 to 25654) from YAC clone (A201F4.3, provided by Dr. Douglas Engel, Northwestern Univ.) covering the human β globin gene region was cloned to a multi-cloning site of pTWV229 vector (TAKARA BIO INC.) (TWV-LCR).

(2) Acceptor Precursor

1.7 kb of fragment of EcoRI-XhoI of pAc-lox71-bsr-pA (provided by Dr. Yamamura, Kumamoto Univ., Kimi Araki, Masatake Araki and Ken-ichi Yamamura (1997)) was inserted into the EcoRI site of pSV2-bsr so as to obtain SV-bsr-lox71. 6 kb of ApaLI fragment of the SV-bsr-lox71 was inserted into the SalI site of pBeloBAC so as to construct BAC-bsr-lox71.

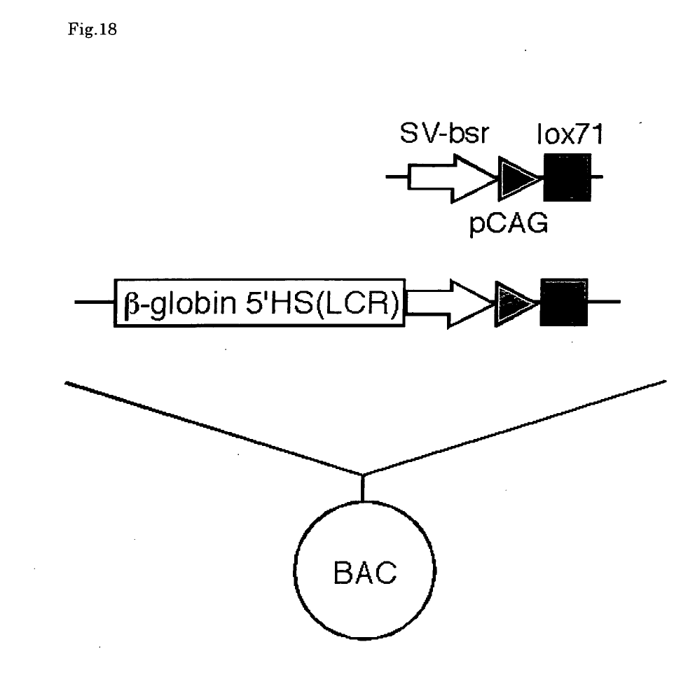

In order to construct a precursor in which β globin LCR (Locus control region, including HS 1 to 5) was added, 20 kb of FspI fragment of TWV-LCR was inserted into the EcoO65I site of BAC-bsr-lox71 (BAC-LCR-lox71, see FIG. 18). Note here that this precursor BAC-LCR-lox71 has a feature that CAG promoter (stable gene expression was expected in various mammalian culture cells and a mouse individual body) was disposed at 5′ side of the lox71 site and CAG selection marker gene was constructed and the expression of gene occurs only when recombinant with respect to a selection marker gene without containing a promoter (promoterless) can be performed as expected at the time of recombination.

(3) Alphoid Precursor

A precursor (Δα50) was constructed by removing SalI-SalI (Cos, loxP sequence) of CMV-a50 (including about 50 kb of alphoid insert (see Example 1) in which alphoid arrays are arranged in tandem).

(4) Donor Plasmid

A 1.2 kb of HindIII-SalI fragment (coding region of puro gene) from pGK-puro (E. coli vector including a PGK promoter, a puro gene, a poly A sequence of a PGK gene, Ampicillin-resistant gene, and replication origin (ori)) and a 3.0 kb of HindIII-XhoI (including lox66) from lox66-Nlaczeo (provided by Dr. Yamamura, Kumamoto Univ., Kimi Araki, Masatake Araki and Ken-ichi Yamamura (1997)) were ligated to each other so as to obtain plox66-puro. 1.2 kb of SpeI-KpnI fragment (lox66-puro cassette) was blunted from plox66-puro and inserted into the HindIII site of pTWV229 (TWV-lox-puro). 1.6 kb of AseI-MluI fragment of pEGFP-C1 (clontech) was blunted and inserted into the SalI site of TWV-lox-puro (Dn-EGFP).

15-2. Construction of Artificial Chromosome Having the Lox Site

The alphoid precursor (Δα50) and the acceptor precursor (BAC-bsr-lox7 or BAC-LCR-lox71) were co-introduced into HT1080 cells, and cell lines containing artificial chromosomes were selected from drug tolerance (bs) cells by FISH.

15-3. Insertion of GFP Gene to Mammalian Artificial Chromosome

To lox15-13 cell lines (containing artificial chromosome having β globin LCR lox71, 2×10⁵), 1 μg of pCAG-Cre (Cre recombinase gene) and 1 μg of Dn-EGFP (lox66 sequence and EGFP gene) were transfected by using lipofectamine plus reagent (Invitrogen). After selection by puromycin, it was confirmed that EGFP inserted by FISH was present on the artificial chromosome.

15-4. Quantification of Expression Amount of EGFP from Artificial Chromosome

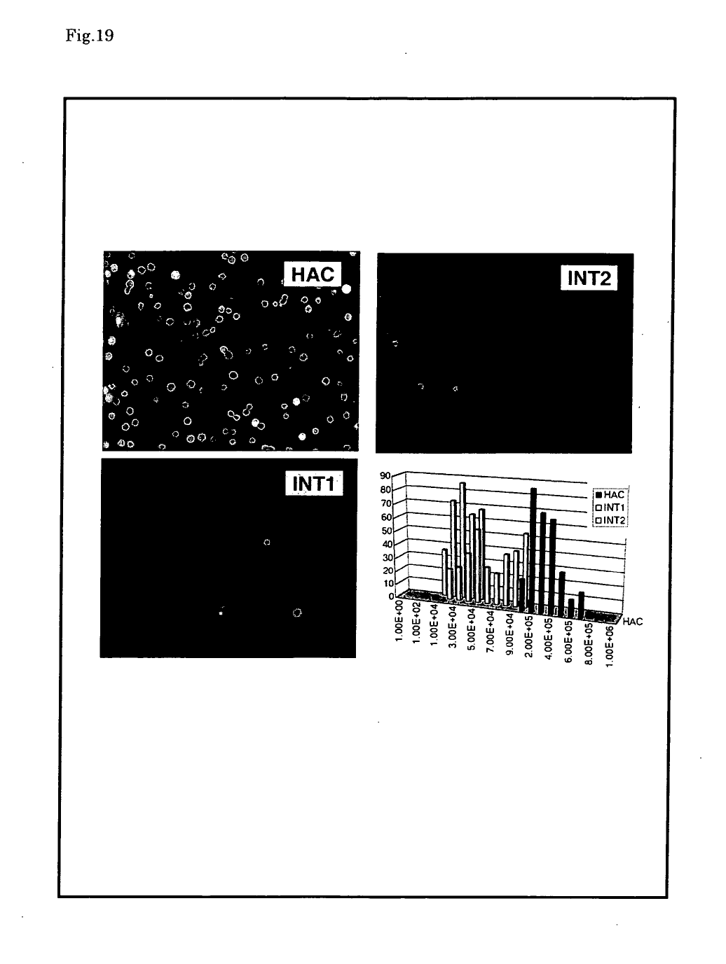

In the case where an accepter precursor that does not contain human β globin LCR (BAC-bsr-lox7lox) was used, since the insertion of Dn-EGFP did not succeed, comparison with stable cell lines, in which pEGFP-C1 (EGFP gene used for production of Dn-EGFP) was incorporated at random on the chromosome, was carried out. The fluorescence intensity of EGFP of individual cells by trypsinization was quantified by the use of IPLab software (NIPPON ROPER Co., Ltd.). As a result, it was shown that the fluorescence of EGFP inserted into the artificial chromosome emitted several times to ten times more than EGFP fluorescence on the chromosome (see FIG. 19).

The present invention is not limited to the description of the above embodiments. A variety of modifications, which are within the scopes of the following claims and which are achieved easily by a person skilled in the art, are included in the present invention.

Document Cited in the Present Description Will be Listed Below

Ando, S., Yang, H., Nozaki, N., Okazaki, T. & Yoda, K. (2002) CENP-A, -B, and -C chromatin complex that contains the I-type alpha-satellite array constitutes the prekinetochore in HeLa cells. Mol. Cell. Biol. 22, 2229-2241.
Bannister, A. J., Zegerman, P., Partridge, J. F., et al. (2001) Selective recognition of methylated lysine 9 on histone H3 by the HP1 chromo domain. Nature 410, 120-124.
Ebersole, T. A., Ross, A., Clark, E., et al. (2000) Mammalian artificial chromosome formation from circular alphoid input DNA does not require telomere repeats. Hum. Mo. Genet. 9, 1623-1631
Dong, J. Y., Fan, P. D. & Frizzell, R. A. (1996) Quantitative analysis of the packaging capacity of recombinant adeno-associated virus. Hum. Gene Ther. 7, 2101-2112.
Festentein, R., Tolaini, M., Corbella, P., et al. (1996) Locus control region function and heterochromatin-induced position effect variegation. Science 271, 1123-1125.
Fisher, K. J., Jooss, K., Alston, J., Yang, Y, Haeker, S. E., High, K., Pathak, R., Raper, S. E. & Wilson, J. M. (1997) Recombinant adeno-associated virus for muscle directed gene therapy. Nature Med. 3, 306-312.
Fournier, R. E. K. & Ruddle, F. H. (1977) Micro-cell-mediated transfer of murine chromosomes into mouse, Chinese hamster, and human somatic cells. Proc. Natl. Acad. Sci. USA 74, 319-323.
Grimes, B. R., Schindelhauer, D., McGill, N. I., et al. (2001) Stable gene expression from a mammalian artificial chromosome. EMBO Rep. 2, 910-914.
Henning, K. A., Novotny, E. A., Compton, S. T., et al. (1999) Human artificial chromosomes generated by modification of a yeast artificial chromosome containing both human alpha-satellite and single-copy DNA sequences. Proc. Natl. Acad. Sci. USA 96, 592-597.
Hibiya, M., Ichinose, H., Ozaki, N., et al. (2000) Normal values and age-dependent changes in GTP cyclohydrolase I activity in stimulated mononuclear blood cells measured by high-performance liquid chromatography. J. Chromatogr B. Biomed. Sci. Appl. 740, 35-42.
Howman, E. V., Fowler, K. J., Newson, A. J., et al. (2000) Early disruption of centromeric chromatin organization in centromere protein A (Cenpa) null mice. Proc. Natl. Acad. Sci. USA 97, 1148-1153.
Hubbard, T., Barker, D., Birney, E., et al. (2002) The Ensembl genome database project. Nucl. Acids Res. 30, 38-41. Ichinose, H., Ohye, T., Takahashi, E., et al. (1994) Hereditary progressive dystonia with marked diurnal fluctuation caused by mutations in the GTP cyclohydrolase I gene. Nature. Genet. 8, 236-242.
Ichinose, H., Ohye, T., Matsuda, Y, et al. (1995) Characterization of mouse and human GTP cyclohydrolase I genes. Mutations in patients with GTP cyclohydrolase I deficiency. J. Biol. Chem. 270, 10062-10071.
Ichinose, H., Suzuki, T., Inagaki, H., Ohye, T. & Nagatsu, T. (1999) Molecular genetics of dopa-responsive dystonia. Biol. Chem. 380, 1355-1364.
Ikeno, M., Masumoto, H. & Okazaki, T. (1994) Distribution of CENP-B boxes reflected in CREST centromere antigenic sites on long-range alpha-satellite DNA arrays of human chromosome 21. Hum. Mol. Genet. 3, 1245-1257.
Ikeno, M., Grimes, B., Okazaki, T., et al. (1998) Construction of YAC-based mammalian artificial chromosomes. Nature Biotechnol. 16, 431-439.
Karpen, G. H. (1994) Position-effect variegation and the new biology of heterochromatin. Curr. Opin. Genet. Dev. 4, 281-291.
Karpen, G. H. & Allshire, R. C. (1997) The case for epigenetic effects on centromere identity and function. Trends Genet. 13, 489-496.
Kaufman, S. (1993) New tetrahydrobiopterin-dependent systems. Annu. Rev. Nutr. 13, 261-286.
Lachner, M., O'Carroll, D., Rea, S., Mechtler, K. & Jenuwein, T. (2001) Methylation of histone H3 lysine 9 creates a binding site for HP1 proteins. Nature 410, 116-120.
Mejia, J. E., Willmott, A., Levy, E., Eamshaw, W. C. & Larin, Z. (2001) Functional complementation of a genetic deficiency with human artificial chromosomes. Am. J. Hum. Genet. 69, 315-326.
Milot, E., Strouboulis, J., Trimborn, T., et al. (1996) Heterochromatin effects on the frequency and duration of LCR-mediated gene transcription. Cell 87, 105-114.
Mineta, T., Rabkin, S. D., Yazaki, T., Hunter, W. D., & Martuza, R. L. (1995) Attenuated multi-mutated herpes simplex virus-1 for the treatment of malignant gliomas. Nature Med. 1, 938-943.
Muramatsu, S., Fujimoto, K., Ikeguchi, K., et al. (2002) Behavioral recovery in a primate model of Parkinson's disease by triple transduction of striatal cells with adeno-associated viral vectors expressing dopamine-synthesizing enzymes. Hum. Gene Ther. 13, 345-354.
Nichol, C. A., Smith, G. K. & Duch, D. S. (1985) Biosynthesis and metabolism of tetrahydrobiopterin and molybdopterin. Annu. Rev. Biochem. 54, 729-764.
Ota, A., Yoshida, S., Nomura, T., Matsui, S., Hagino, Y, Umezawa, K., Katoh, S. & Nagatsu, T. (1996) Tetrahydrobiopterin biosynthesis enhanced by lipopolysaccharide stimulation in murine neuroblastoma cell line N1E-115. J. Neurochem. 67, 2540-2548.
Palmer, D. K., O'Day, K., Trong, H. L., Charbonneau, H. & Margolis, R. L. (1991) Purification of the centromere-specific protein CENP-A and demonstration that it is a distinctive histone. Proc. Natl. Acad. Sci. USA 88, 3734-3738.
Pfeifer, A. & Verma, I. M. (2001) Gene therapy: promises and problems. Annu. Rev. Genomics Hum. Genet. 2, 177-211
Platero, J. S., Hartnett, T. & Eissenberg, J. C. (1995) Functional analysis of the chromo domain of HP1. EMBO J. 14, 3977-3986.
Shen, M. H., Yang, J., Loupart, M. L., Smith, A., Brown, W. (1997) Human mini-chromosomes in mouse embryonal stem cells. Hum. Mol. Genet. 6, 1375-82.
Tanaka, K., Kaufman, S. & Milstein, S. (1989) Tetrahydrobiopterin, the cofactor for aromatic amino acid hydroxylases, is synthesized by and regulates proliferation of erythroid cells. Proc. Natl. Acad. Sci. USA 86, 5864-5867.
Wade-Martins, R., White, R. E., Kimura, H., Cook, P. R. & James M. R. (2000) Stable correction of a genetic deficiency in human cells by an episome carrying a 115 kb of genomic transgene. Nature Biotechnol. 18, 1311-1314.
Werner, E. R., Werner-Felmayer, G., Fuchs, D., et al. (1990) Tetrahydrobiopterin biosynthetic activities in human macrophages, fibroblasts, THP-1, and T 24 cells. GTP-cyclohydrolase I is stimulated by interferon-gamma, and 6-pyruvoyl tetrahydropterin synthase and sepiapterin reductase are constitutively present. J. Biol. Chem. 265, 3189-3192.
Werner, E. R., Werner-Felmayer, G. & Wachter, H. (1993) Tetrahydrobiopterin and cytokines. Proc. Soc. Exp. Biol. Med. 203, 1-12.
Yen, T. J., Compton, D. A., Wise, D., et al. (1991) CENP-E, a novel human centromere-associated protein required for progression from metaphase to anaphase. EMBO J. 10, 1245-1254.
Kimi Araki, Masatake Araki and Ken-ichi Yamamura. Target integration of DNA using munatn lox sites in embryonic stem cells. Nucleic Acids Research, 1997, vol. 25, No 4, 868-872

### INDUSTRIAL APPLICABILITY

The present invention provides a mammalian artificial chromosome containing a huge DNA region including an original regulatory region in addition to a gene of interest. Therefore, gene expression from the gene contained in the mammalian artificial chromosome can be carried out in an original regulation system.

The mammalian artificial chromosome of the present invention can be used also for transferring itself to the other cells, or also can be used for study at the individual body level by way of human embryonic stem cells, etc. Therefore, it is an extremely useful tool for study of tissue specific gene expression and gene expression over time, study of human-type genes using a model animal, development of drugs (inhibitors, promoters, etc.), and the like.

For example, by using the embryonic stem cell containing an artificial chromosome with a gene of interest obtained by the method of the present invention, transformed animals (including chimeric animals) containing an artificial chromosome expressing a gene of interest can be produced, thus enabling the analysis of expression system of the single gene at the individual level. Furthermore, it is though that a clone animal carrying HAC of the present invention can be produced. The transformed animal containing the above-mentioned human artificial chromosome can be used as a model for gene therapy. Furthermore, it can be also used for analyzing the effect of drug on the target gene under physiological conditions.

The mammalian artificial chromosome of the present invention is useful as a vector for gene therapy. Thus, the mammalian artificial chromosome of the present invention provides a simple and general method of transporting a huge DNA region including the original regulatory region in addition to a gene of interest.

1. A production method of a mammalian artificial chromosome, comprising:
a first step of introducing a first vector being circular in form and comprising a mammalian centromere sequence and a second vector being circular in form and comprising an insertion sequence for specifically inserting a sequence of interest and an insulator sequence into a mammalian host cell;
a second step of selecting transformed cells; and
a third step of selecting a cell containing a mammalian artificial chromosome from the selected transformed cells.

2. A production method of a mammalian artificial chromosome, comprising:
a first step of introducing a first vector consisting of a yeast artificial chromosome having a mammalian centromere sequence and a mammalian telomere sequence and a second vector consisting of a yeast artificial chromosome having an insertion sequence for specifically inserting a sequence of interest and an insulator sequence into a mammalian host cell;
a second step of selecting transformed cells; and
a third step of selecting a cell containing a mammalian artificial chromosome from the selected transformed cells.

3. The production method according to claim 1 or 2, wherein the first vector has a selection marker gene and the selection of the transformed cells in the second step is carried out by using the selection marker gene.

4. The production method according to any of claims 1 to 3, wherein the mammalian centromere sequence comprises a region in which a plurality of the following sequences are arranged at regular intervals:
5′-NTTCGNNNNANNCGGGN-3′: SEQ ID NO. 1, wherein N is selected from the group consisting of A, T, C and G.

5. The production method according to any of claims 1 to 4, wherein the mammalian centromere sequence comprises a sequence derived from a human chromosome alpha satellite region.

6. The production method according to claim 5, wherein the mammalian centromere sequence comprises a 11mer repeat unit derived from a human chromosome 21.

7. The production method according to any of claims 1 to 6, wherein the size of the mammalian centromere sequence is about 50 kb or less.

8. (canceled)

9. (canceled)

10. (canceled)

11. (canceled)

12. (canceled)

13. The production method according to any of claims 1 to 7, wherein the insertion sequence is a loxP site, a FRT site, or a sequence obtained by partial modification of a loxP site or a FRT site and has a function for inserting the sequence of interest.

14. The production method according to any of claims 1 to 13, wherein the quantity ratio of the first vector to the second vector, which are inserted in the first step, is in the range from about 10:1 molecular ratio to about 1:10 molecular ratio.

15. (canceled)

16. (canceled)

17. A mammalian artificial chromosome obtainable by the production method described in any of claims 1 to 16,
which comprises a mammalian replication origin, a mammalian centromere sequence, an insertion sequence for specifically inserting a sequence of interest and an insulator sequence; and
which is circular in form and is replicated in a mammalian cell, maintained extrachromosomally in a host cell, and transmitted to daughter cells during cell division.

18. A mammalian artificial chromosome obtainable by the production method described in any of claims 1 to 16,
which comprises a mammalian replication origin, a mammalian centromere sequence, a mammalian telomere sequence, an insertion sequence for specifically inserting a sequence of interest and an insulator sequence; and
which is linear in form and is replicated in a mammalian cell, maintained extrachromosomally in a host cell, and transmitted to daughter cells during cell division.

19. (canceled)

20. (canceled)

21. (canceled)

22. (canceled)

23. A mammalian artificial chromosome,
which comprises a mammalian replication origin, a mammalian centromere sequence, an insertion sequence for specifically inserting a sequence of interest and an insulator sequence, and
which is circular in form and is replicated in a mammalian cell, maintained extrachromosomally in a host cell, and transmitted to daughter cells during cell division.

24. A mammalian artificial chromosome,
which comprises a mammalian replication origin, a mammalian centromere sequence, a mammalian telomere sequence, an insertion sequence for specifically inserting a sequence of interest and an insulator sequence,
which is linear in form and is replicated in a mammalian cell, maintained extrachromosomally in a host cell, and transmitted to daughter cells during cell division.

25. The mammalian artificial chromosome according to claim 23 or 24, wherein the insertion sequence is a loxP site, a FRT site, or a sequence obtained by partial modification of a loxP site or a FRT site and has a function for inserting the sequence of interest.

26. The mammalian artificial chromosome according to any of claims 17 to 25, wherein the mammalian centromere sequence comprises a region in which a plurality of the following sequences are arranged at regular intervals:
5′-NTTCGNNNNANNCGGGN-3′: SEQ ID NO. 1, wherein N is selected from the group consisting of A, T, C and G.

27. The mammalian artificial chromosome according to any of claims 17 to 25, wherein the mammalian centromere sequence comprises a sequence derived from a human chromosome alpha satellite region.

28. The mammalian artificial chromosome according to claim 27, wherein the mammalian centromere sequence comprises an 11mer repeat unit derived from a human chromosome 21.

29. The mammalian artificial chromosome according to any of claims 17 to 28, comprising a plurality of the functional sequences or the insertion sequences.

30. (canceled)

31. A mammalian cell containing the mammalian artificial chromosome described in any of claims 17 to 30 outside the autonomous chromosome.

32. A human cell containing the mammalian artificial chromosome described in any of claims 17 to 30 outside the autonomous chromosome.

33. An embryonic stem cell containing the mammalian artificial chromosome described in any of claims 17 to 30 outside the autonomous chromosome.

34. A production method of a mammalian cell in which the functional sequence or the insertion sequence is introduced in a state in which they can be maintained stably for a long term, the method comprising:
introducing the mammalian artificial chromosome obtained by the production method described in any of claims 1 to 16 or the mammalian artificial chromosome described in any of claims 17 to 30 into mammalian cells as target cells.

35. A production method of a mammalian cell containing a mammalian artificial chromosome, the method comprising:
a first step of introducing a first vector being circular in form and comprising a mammalian centromere sequence and a second vector being circular in form and comprising an insertion sequence for specifically inserting a sequence of interest and an insulator sequence into mammalian host cells;
a second step of selecting transformed cells;
a third step of selecting a cell containing a mammalian artificial chromosome from the selected transformed cells;
a fourth step of isolating the mammalian artificial chromosome from the selected cells; and
a fifth step of introducing the isolated mammalian artificial chromosome into a mammalian cell as a target cell.

36. A production method of a mammalian cell containing a mammalian artificial chromosome, the method comprising:
a first step of introducing a first vector consisting of a yeast artificial chromosome having a mammalian centromere sequence and a mammalian telomere sequence and a second vector consisting of a yeast artificial chromosome having an insertion sequence for specifically inserting a sequence of interest and an insulator sequence into mammalian host cells;
a second step of selecting transformed cells;
a third step of selecting a cell containing a mammalian artificial chromosome from the selected transformed cells;
a fourth step of isolating the mammalian artificial chromosome from the selected cell; and
a fifth step of introducing the isolated mammalian artificial chromosome into a mammalian cell as a target cell.

37. A production method of a micro-cell containing a mammalian artificial chromosome, the method comprising:
a first step of introducing a first vector being circular in form and comprising a mammalian centromere sequence and a second vector being circular in form and comprising an insertion sequence for specifically inserting a sequence of interest and an insulator sequence into mammalian host cells;
a second step of selecting transformed cells;
a third step of selecting a cell containing a mammalian artificial chromosome from the selected transformed cells;
a fourth step of fusing the selected cell with a mammalian cell having an ability of forming micro-cells;
a fifth step of selecting a hybrid cell capable of forming micro-cells and containing the mammalian artificial chromosome; and
a sixth step of forming micro-cells from the selected hybrid cell.

38. A production method of a micro-cell containing a mammalian artificial chromosome, the method comprising:
a first step of introducing a first vector consisting of a yeast artificial chromosome including a mammalian centromere sequence and a mammalian telomere sequence and a second vector consisting of a yeast artificial chromosome including an insertion sequence for specifically inserting a sequence of interest and an insulator sequence into mammalian host cells;
a second step of selecting transformed cells;
a third step of selecting a cell containing a mammalian artificial chromosome from the selected transformed cells;
a fourth step of fusing the selected cell with a mammalian cell having an ability of forming micro-cells;
a fifth step of selecting a hybrid cell having an ability of forming micro-cells and containing a mammalian artificial chromosome; and
a sixth step of forming micro-cells from the selected hybrid cell.

39. A production method of mammalian cells containing a mammalian artificial chromosome, comprising:
fusing the micro-cell obtainable by the production method described in claim 37 or 38 with a mammalian cell as a target cell.

40. A production method of a mammalian cell containing a mammalian artificial chromosome, comprising:
isolating the mammalian artificial chromosome from the host cell containing the mammalian artificial chromosome described in any of claims 17 to 30; and
introducing the isolated mammalian artificial chromosome into a mammalian cell as a target cell.

41. A production method of a micro-cell containing a mammalian artificial chromosome, the method comprising:
fusing a host cell containing the mammalian artificial chromosome described in any of claims 17 to 30 and a mammalian cell having an ability of forming micro-cells;
selecting a hybrid cell having an ability of forming micro-cellsi and containing the mammalian artificial chromosome; and
forming micro-cells from the selected hybrid cells.

42. A production method of a mammalian cell containing a mammalian artificial chromosome, the method comprising:
fusing the micro-cell obtainable by the production method described in claim 41 with a mammalian cell as a target.

43. The production method of a mammalian cell according to any of claims 34, 35, 36, 39, 40 and 42, wherein the mammalian cell as a target cell is an embryonic stem cell, embryonic germ cell, or tissue stem cell.

44. The production method of a mammalian cell according to any of claims 34, 35, 36, 39, 40 and 42, wherein the mammalian cell as a target cell is formed by inducing an embryonic stem cell, embryonic germ cell, or tissue stem cell so as to be differentiated to a cell of specific tissue.

45. The production method of a mammalian cell according to any of claims 34, 35, 36, 39, 40 and 42, wherein the mammalian cell as a target cell is a fertilized egg.

46. (canceled)

47. (canceled)

48. (canceled)

49. (canceled)

50. A vector used for producing a mammalian artificial chromosome, comprising: a sequence of a loxP site or FRT site, or a sequence obtainable by partial modification of a loxP site or FRT site, the sequence having a function for inserting the sequence of interest, and an insulator sequence.

51. (canceled)

52. A non-human transformed animal into which the mammalian artificial chromosome described in any of claims 17 to 19 is introduced.

53. (canceled)

54. An XO type mouse embryonic stem into which a mammalian artificial chromosome described in any of claims 17 to 19 is introduced

55. (canceled)

56. A female chimeric mouse into which a mammalian artificial chromosome described in any of claims 17 to 19 is introduced.


SEQUENCE LISTING (23 sequences)

SEQ ID NO: 1 - DNA, 17 bp, Homo sapiens
  source (1)..(17): Human chromosome centromere region
  misc_feature (1)..(1): n stands for any base
  misc_feature (6)..(9): n stands for any base
  misc_feature (11)..(12): n stands for any base
  misc_feature (17)..(17): n stands for any base
nttcgnnnnanncgggn

SEQ ID NO: 2 - DNA, 17 bp, Homo sapiens
  source (1)..(17): Human chromosome 21 centromere region
  misc_feature (1)..(1): n stands for any base
nttcgttggaaacggga

SEQ ID NO: 3 - DNA, 1868 bp, Homo sapiens
  source (1)..(1868): Human chromosome 21 centromere region
aattcaaataaaaggtagacagcagcattctcagaaatttctttctgatgtctgcattcaactcatagagttgaagattgcctttcatagagcaggtttgaaacactctttctggagtatctggatgtggacatttggagcgctttgatgcctacggtggaaaagtaaatatcttccataaaaacgagacagaaggattctcagaaacaagtttgtgatgtgtgtactcagctaacagagtggaacctttctttttacagagcagctttgaaactctatttttgtggattctgcaaattgatatttagattgctttaacgatatcgttggaaaagggaatatcgtcatacaaaatctagacagaagcattctcacaaacttctttgtgatgtgtgtcctcaactaacagagttgaacctttcttttgatgcagcagtttggaaacactctttttgtagaaactgtaagtggatatttggatagctctaacgatttcgttggaaacgggaatatcatcatctaaaatctagacagaagcactattagaaactacttggtgatatctgcattcaagtcacagagttgaacattcccttactttgagcacgtttgaaacactcttttggaagaatctggaagtggacatttggagcgctttgactgcctttgttgaaaaggaaacgtcttccaataaaagccagacagaagcattctcagaaacttgttcgtgatgtgtgtactcaactaaaagagttgaacctttctattgatagagcagttttgaaacactctttttgtggattctgcaagtggatatttggattgctttgaggatttcgttggaagcgggaattcgtataaaaactagacagcagcattcccagaaatttctttcggatatttccattcaactcatagagatgaacatggcctttcatagagcaggtttgaaacactctttttgtagtttgtggaagtggacatttcgatcgccttgacgcctacggtgaaaaaggaaatatcttcccataaaaatagacagaagcattctcagaaacttgttggtgatatgtgtctcaactaacagagttgaactttgccattgatagagagcagttttgaaacactctttttgtggaatctgcaagtggatatttggatagcttggaggatttcgttggaagcgggaattcaaataaaaggtagacagcagcattctcagaaatttctttctgatgactgcattcaactcatagagttgaacattccctttcatagagcaggtttgaaacactctttctggagtatctggatgtggacatttggagcgctttgatgcctatggtgaaaaagtaaatatcttcccataaaaacgagacagaaggattctgagaaacaagtttgtgatgtgtgtactcagctaacagagtggaacctctcttttgatgcagcagtttggaaacactctttttgtagaaactgtaagtggatatttggatagctctaatgatttcgttggaaacgggaatatcatcatctaaaatctagacagaagccctctcagaaactactttgtgatatctgcattcaagtcacagagttgaacattcgctttcttagagcacgttggaaacactctttttgtagtgtctggaagtggacatttggagcgctttgatgcctttggtgaaaaagggaatgtcttcccataaaaactagacagaagcattctcagaaacttgtttttgatgtgtgtacccagccaaaggagttgaacatttctattgatagagcagttttgaaacactctttttgtggaaaatgcaggtggatatttggatagcttggaggatttcgttggaagcggg

SEQ ID NO: 4 - DNA, 8286 bp, Artificial Sequence
  Description of Artificial SequenceProbe for an arm region of YAC
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcaattccctttagtataaatttcactctgaaccatcttggaaggaccggtaattatttcaaatctctttttcaattgtatatgtgttatgttatgtagtatactctttcttcaacaattaaatactctcggtagccaagttggtttaaggcgcaagactttaatttatcactacggaattccgtaatcttgagatcgggcgttcgatcgccccgggagatttttttgttttttatgtcttccattcacttcccagacttgcaagttgaaatatttctttcaagggaattgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatccccccccccctttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattactcgggcgtaatgatttttataatgacgaaaaaaaaaaaattggaaagaaaagggggggggggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagccaagcttatccctcgagggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaaatgaaatgagatgatacttgcttatctcatagttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccaga

SEQ ID NO: 5 - DNA, 3631 bp, Homo sapiens
aagaccagatagtacagggcctggctacaaaaatacaagcttttactatgctattgcaatactaaacgataagcattaggatgttaagtgactcaggaaataagattttgggaaaaagtaatctgcttatgtgcacaaaatggattcaagtttgcagataaaataaaatatggatgatgattcaaggggacagatacaatggttcaaacccaagaggagcagtgagtctgtggaattttgaaggatggacaaaggtggggtgagaaagacatagtattcgacctgactgtgggagatgagaaggaagaaggaggtgataaatgactgaaagctcccagactggtgaagataacaggaggaaaccatgcacttgaccctggtgactctcatgtgtgaagggtagagggatattaacagatttactttttaggaagtgctagattggtcagggagttttgaccttcaggtcttgtgtctttcatatcaaggaacctttgcattttccaagttagagtgccatattttggcaaatataactttattagtaattttatagtgctctcacattgatcagactttttcctgtgaattacttttgaatttggctgtatatatccagaatatgggagagagacaaataattattgtagttgcaggctatcaacaatactggtctctctgagccttataacctttcaatatgccccataaacagagtaaacagggattattcatggcactaaatattttcacctaggtcagtcaacaaatggaggcaatgtgcattttttgatacatatttttatatatttatggggcatgtgatacttacatgcctagaacatgtgactgattaagtctagatatttaggatatccattactttgagcatttatcatttctatgtattgagaaaatttcaaatcctcatttctgaccattttgaaatatataataaatagtaattaactatagtcaccctactcaaatatcaacattataaactaactaatccttctttccacttttttaccaaccaacatctcttaaatcccctgccatacacatcacacatttttcagctctgataactatcattctactctcataccaccatgagaccacttttttagctccacagatgaataaaaacatgtgatatttgactttctgtatctggcttattttattatctatctctttggcataccaagagtttgtttttgttctgcttcagggctttcaattaacataatgacctctggttccatccatgttgctacaaatgacaagatttcattctttttcatggcaaaatagtactgtgcaaaaaatacaattttttaatccgttcatctgttgatagacacttaggttgatcccaaaccttaactattgtgaataggtgcttcaataaacatgagtgtaatgtgtccattggatatactgatttcctttcttttggataaataaccactagtgagattgctggattgtatgatagttctgtttttagtttattgagaaatcttcatactgttttccataatggttgtactattttacattcccaccaacagtgtgtaagaaagagttcccttttctccatatcctcacaaggatctgttattttttgtcttttttgttaatagcattttaactagagtaagtagatatctcattgtagttttgatttgcatttccctgatcattagtgatgttgagattttttcatatgtttgttggtcatttgtatatctttttctgagattgtctgttcatgtccttatcctacttttattgggattgttgttattttcttgataatcattgtgtcattttagagcctggatattattcttttgtcagatgtatagattgtgaagattttctcctctgtgggttgtctgtttattctgcagactcttccttttgccatgcaaaagctctttagtttaatttagtcccagatattttctttgtttttatgtgtttgcatttgtgttcttgtcatgaaatcctttcctaagccaatgtgtagaagggtttttccgatgttattttctagaattgttacagtttcaggcttagatttaagtccttgatccatcttaagttgatttttgtataaggtgagagatgaagatccagtttcattctcctacatgtagcttgccagctatcccgactcatttgttgaatagggtgccctttcccatttatgtttttgtttgctttgtcaaagatcagttcggatgtaagtatttgagtttatttctgggttctctattctgttccattggtccgatgtgcctatttgtacaccagcatcatgctgtgtttttggtgactatggccttattgtatagtttgaaatgaggtaatgtaatgccattcagatttgttcttttttttagacttgcttgtttattgggctcttttttggttccataagaattttaggattgttttttctagttctgtgaaggctaatggtggtatttatgggaattgcaatgcaatttgtaggttgcttctggcattatggccattttcacaatattgattctacccatctatgagaatggcatgtgtttccatttgtttgtgtcttatatgattactatcagccgtgttttgtagttttccttgtagatgtctttcacctccttggttaggtatatattcctaagtttttgttttgttttgttttgttttttgcagctattgtaaaaggggttgagttattgattttattctcatcttggtcattgctggtatgtaagaaagcaactcattggtgtacgttaattttgtatccagaaactttgctgaattattttatcagttctagggggttttggaggagtctttagagttttctacatacacaatcatatcatcagcaaacagtgacagtttgactttctctttaacaatttggatgtgctttacttgtttctcttgtctgattgctcttgctaggacttccagtaatatgttaaagagaagtggtgagagtgggtatccttgtctcattccagttttcagacagaatgcttttaactttttcccattcaatataatgttggctgtgtgtttaccatagctggcttttattacattgaggtatgtcctttgtaaaccgattttgctgagttttagtcataaagtgatgttgaattttgttgaatgcagtttctgtggctattgagataatcacatgatttttgtttccaattctctttatgttgtgtatcacacttattgacttgcgtatgttaaaccatccgtgcatccctcgcatgaaaccacttgatcatgggttttgatatgccgtgtgggatgctattagctatattttgtcaaggatgttggcatctatgttcatcagggatattgatctgtagtgttttttttttttggttatgttctttcccagttttggtattaaggtgatactggcttcatagaatgatttagggaggattctctctttctctatcttgtagaatactgtcaataggattggtatcaattcttctttgaatgtctggtagaattcgaacgtctcctttaggttttctagtttattcatgtaaaggtgttcatagtaaccttgaataatctt

SEQ ID NO: 6 - DNA, 3386 bp, Homo sapiens
tgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtcagcgtgtgtttcttttaacgtcttcagcctacaacatacagggttcatggtgggaagaagatagcaagatttaaattatggccagtgactagtgcttgaaggggaacaactacctgcatttaatgggaaggcaaaatctcaggctttgagggaagttaacataggcttgattctgggtggaagctgggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagcctcttgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgttggagaaatccggtgggaagaaagacctctataggacaggacttctcagaaacagatgttttggaagagatgggaaaaggttcagtgaagacctgggggctggattgattgcagctgagtagcaaggatggttcttaatgaagggaaagtgttccaagctttaggaattcaaggtttagtcaggtgtagcaattctattttattaggaggaatactatttctaatggcacttagcttttcacagcccttgtggatgcctaagaaagtgaaattaatcccatgccctcaagtgtgcagattggtcacagcatttcaagggagagacctcattgtaagactctgggggaggtggggacttaggtgtaagaaatgaatcagcagaggctcacaagtcagcatgagcatgttatgtctgagaaacagaccagcactgtgagatcaaaatgtagtgggaagaatttgtacaacattaattggaaggtttacttaatggaatttttgtatagttggatgttagtgcatctctataagtaagagtttaatatgatggtgttacggacctggtgtttgtgtctcctcaaaattcacatgctgaatccccaactcccaactgaccttatctgtgggggaggcttttgaaaagtaattaggtttagctgagctcataagagcagatccccatcataaaattattttccttatcagaagcagagagacaagccatttctctttcctcccggtgaggacacagtgagaagtccgccatctgcaatccaggaagagaaccctgaccacgagtcagccttcagaaatgtgagaaaaaactctgttgttgaagccacccagtcttttgtattttgttatagcaccttacactgagtaaggcagatgaagaaggagaaaaaaataagcttgggttttgagtgaactacagaccatgttatctcaggtttgcaaagctcccctcgtcccctatgtttcagcataaaatacctactctactactctcatctataagacccaaataataagcctgcgcccttctctctaactttgatttctcctatttttacttcaacatgctttactctagccttgtaatgtctttacatacagtgaaatgtaaagttctttattctttttttctttctttcttttttctcctcagcctcagaatttggcacatgcccttccttctttcaggaacttctccaacatctctgcctggctccatcatatcataaaggtcccacttcaaatgcagtcactaccgtttcaggatatgcactttctttcttttttgttttttgttttttttaagtcaaagcaaatttcttgagagagtaaagaaataaacgaatgactactgcataggcagagcagccccgagggccgctggttgttccttttatggttatttcttgatgatatgttaaacaagttttggattatttatgccttctctttttaggccatatagggtaactttctgacattgccatggcatgtttcttttaatttaatttactgttaccttaaattcaggggtacacgtacaggatatgcaggtttgttttataggtaaaagtgtgccatggttttaatgggttttttttttcttgtaaagttgtttaagtttcttgtttactctggatattggcctttgtcagaagaatagattggaaaatctttttcccattctgtagattgtctttcgctctgatggtagtttcttttgctgagcaggagctctttagtttaattagattccattggtcaatttttgcttttgctgcaattgcttttcacgctttcatcatgaaatctgtgcccgtgtttatatcatgaatagtattgccttgatttttttctaggctttttatagtttggggtttttcatttaagtctctaatccatccggagttaattttggataaggtataaggaaggagtccagtttcatttttcagcatatggctagccagttctcccccatcatttattaaattgaaaatcctttccccattgcttgcttttgtcaggtttctaaaagacagatggttgtaggtacaatatgcagtttcttcaagtcatataataccatctgaaatctcttattaattcatttcttttagtatgtatgctggtctcctctgctcactatagtgagggcaccattagccagagaatctgtctgtctagttcatgtaagattctcagaattaagaaaaatggatggcatatgaatgaaacttcatggatgacatatggaatctaatgtgtatttgttgaattaatgcataagatgcaacaagggaaaggttgacaactgcagtgataacctggtattgatgatataagagtctatagatcacagtagaagcaataatcatggaaaacaattggaaatggggaacagccacaaacaagaaagaatcaatactaccaggaaagtgactgcaggtcacttttcctggagcgggtgagagaaaagtggaagttgcagtaactgccgaattcctggttggctgatggaaa

SEQ ID NO: 7 - DNA, 2838 bp, Homo sapiens
gtgtaagaaggttcctgaggctctacagatagggagcacttgtttattttacaaagagtacatgggaaaagagaaaagcaagggaaccgtacaaggcattaatgggtgacacttctacctccaaagagcagaaattatcaagaactcttgatacaaagataatactggcactgcagaggttctagggaagacctcaaccctaagacatagcctcaagggtaatagctacgattaaactccaacaattactgagaaaataatgtgctcaattaaaggcataatgattactcaagacaatgttatgttgtctttcttcctccttcctttgcctgcacattgtagcccataatactataccccatcaagtgttcctgctccaagaaatagcttcctcctcttacttgccccagaacatctctgtaaagaatttcctcttatcttcccatatttcagtcaagattcattgctcacgtattacttgtgacctctcttgaccccagccacaataaacttctctatactacccaaaaaatctttccaaaccctccccgacaccatatttttatatttttcttatttatttcatgcacacacacacactccgtgctttataagcaattctgcctattctctaccttcttacaatgcctactgtgcctcatattaaattcatcaatgggcagaaagaaaatatttattcaagaaaacagtgaatgaatgaacgaatgagtaaatgagtaaatgaaggaatgattattccttgctttagaacttctggaattagaggacaatattaataataccatcgcacagtgtttctttgttgttaatgctacaacatacaaagaggaagcatgcagtaaacaaccgaacagttatttcctttctgatcataggagtaatatttttttccttgagcacatttttgccataggtaaaattagaaggatttttagaactttctcagttgtatacatttttaaaaatctgtattatatgcatgttgattaattttaaacttacttgaatacctaaacagaatctgttgtttccttgtgtttgaaagtgctttcacagtaactctgtctgtactgccagaatatactgacaatgtgttatagttaactgttttgatcacaacattttgaattgactggcagcagaagctctttttatatccatgtgttttccttaagtcattatacatagtaggcatgagactctttatactgaataagatatttaggaaccactggtttacatatcagaagcagagctactcagggcattttggggaagatcactttcacattcctgagcatagggaagttctcataagagtaagatattaaaaggagatacttgtgtggtattcgaaagacagtaagagagattgtagaccttatgatcttgatagggaaaacaaactacattcctttctccaaaagtcaaaaaaaaagagcaaatatagcttactataccttctattcctacaccattagaagtagtcagtgagtctaggcaagatgttggccctaaaaatccaaataccagagaattcatgagaacatcacctggatgggacatgtgccgagcaacacaattactatatgctaggcattgctatcttcatattgaagatgaggaggtcaagagatgaaaaaagacttggcaccttgttgttatattaaaattatttgttagagtagagcttttgtaagagtctaggagtgtgggagctaaatgatgatacacatggacacaaagaatagatcaacagacacccaggcctacttgagggttgagggtgggaagagggagacgatgaaaaagaacctattgggtattaagttcatcactgagtgatgaaataatctgtacatcaagacccagtgatatgcaatttacctatataacttgtacatgtacccccaaatttaaaataaagttaaaacaaagtataggaatggaattaattcctcaagatttggctttaattttatttgataatttatcaaatggttgtttttcttttctcactatggcgttgctttataaactatgttcagtatgtctgaatgaaagggtgtgtgtgtgtgtgaaagagagggagagaggaagggaagagaggacgtaataatgtgaatttgagttcatgaaaatttttcaataaaataatttaatgtcaggagaattaagcctaatagtctcctaaatcatccatctcttgagcttcagagcagtcctctgaattaatgcctacatgtttgtaaagggtgttcagactgaagccaagattctacctctaaagagatgcaatctcaaatttatctgaagactgtacctctgctctccataaattgacaccatggcccacttaatgaggttaaaaaaaagctaattctgaatgaaaatctgagcccagtggaggaaatattaatgaacaaggtgcagactgaaatataaattttctgtaataattatgcatatactttagcaaagttctgtctatgttgactttattgcttttggtaagaaatacaactttttaaagtgaactaaactatcctatttccaaactattttgtgtgtgtgcggtttgtttctatgggttctggttttcttggagcatttttatttcattttaattaattaattctgagagctgctgagttgtgtttactgagagattgtgtatctgcgagagaagtctgtagcaagtagctagactgtgcttgacctaggaacatatacagtagattgctaaaatgtctcacttggggaattttagactaaacagtagagcatgtataaaaatactctagtcaagtgctgctt

SEQ ID NO: 8 - DNA, 6 bp, Homo sapiens
ttaggg

SEQ ID NO: 9 - DNA, 1884 bp, Homo sapiens
gtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggtctttatgaaaattgttttcactacactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctacagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgagattatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtcagcgtgtgtttcttttaacgtcttcagcctaca

SEQ ID NO: 10 - DNA, 28 bp, Artificial Sequence
  Description of Artificial SequencePCR primer
aagaccagatagtacagggcctggctac

SEQ ID NO: 11 - DNA, 28 bp, Artificial Sequence
  Description of Artificial SequencePCR primer
aagattattcaaggttactatgaacacc

SEQ ID NO: 12 - DNA, 38 bp, Artificial Sequence
  Description of Artificial SequencePCR primer
tgctaatgcttcatctagaaacttatatcctttaattc

SEQ ID NO: 13 - DNA, 35 bp, Artificial Sequence
  Description of Artificial SequencePCR primer
tttccactcgagccaaccaggaattcggcagttac

SEQ ID NO: 14 - DNA, 38 bp, Artificial Sequence
  Description of Artificial SequencePCR primer
gtgtaagaaggttctctagaggctctacagatagggag

SEQ ID NO: 15 - DNA, 39 bp, Artificial Sequence
  Description of Artificial SequencePCR primer
aagcagcacttgactcgagtatttttatacatgctctac

SEQ ID NO: 16 - DNA, 23 bp, Artificial Sequence
  Description of Artificial SequencePCR primer
gtatacatacatacctgaatatg

SEQ ID NO: 17 - DNA, 28 bp, Artificial Sequence
  Description of Artificial SequencePCR primer
tgtaggctgaagacgttaaaagaaacac

SEQ ID NO: 18 - DNA, 22 bp, Artificial Sequence
  Description of Artificial SequencePrimer for RT-PCR
gatgccataaagcacctggatg

SEQ ID NO: 19 - DNA, 24 bp, Artificial Sequence
  Description of Artificial SequencePrimer for RT-PCR
ttgcagaataaagcctatccttga

SEQ ID NO: 20 - DNA, 25 bp, Artificial Sequence
  Description of Artificial SequencePrimer for RT-PCR
tcacccacactgtgcccatctacga

SEQ ID NO: 21 - DNA, 25 bp, Artificial Sequence
  Description of Artificial SequencePrimer for RT-PCR
cagcggaaccgctcattgccaatgg

SEQ ID NO: 22 - DNA, 21 bp, Artificial Sequence
  Description of Artificial SequencePCR primer
catcgtctctctgaaaaatcg

SEQ ID NO: 23 - DNA, 21 bp, Artificial Sequence
  Description of Artificial SequencePCR primer
aggaaacagcaaaactgtgac